(19) United States
(12) Patent Application Publication
(10) Pub. No.: US 2026/0337618 A1
(43) Pub. Date: Oct. 29, 2026

(54) HOMOTRIMERIC APTAMER FOR MOLECULAR RECOGNITION, METHODS OF MAKING AND USES THEREOF

(71) Applicant: MCMASTER UNIVERSITY, Hamilton (CA)

(72) Inventors: Yingfu LI, Dundas (CA); John BRENNAN, Dundas (CA); Jiuxing LI, Hamilton (CA); Zijie ZHANG, Hamilton (CA); Jimmy GU, Mississauga (CA); Ryan AMINI, Hamilton (CA); Alexandria MANSFIELD, Hamilton (CA); Jianrun XIA, Ancaster (CA); Dawn WHITE, Barrie (CA); Hannah D. STACEY, St. Marys (CA); Jann C. ANG, Mississauga (CA); Gurpreet PANESAR, Hamilton (CA); Alfredo CAPRETTA, Dundas (CA); Carlos FILIPE, Ancaster (CA); Karen MOSSMAN, Ancaster (CA); Bruno J. SALENA, Ancaster (CA); Cynthia BALION, Hamilton (CA); Leyla SOLEYMANI, Oakville (CA); Matthew S. MILLER, Hamilton (CA); Deborah YAMAMURA, Burlington (CA); Sam AFKHAMI, Oakville (CA); Michael Rein D'AGOSTINO, Toronto (CA)

(73) Assignee: MCMASTER UNIVERSITY, Hamilton, ON (CA)

(21) Appl. No.: 19/113,862

(22) PCT Filed: Sep. 22, 2023
(86) PCT No.: PCT/CA2023/051255

§ 371 Date: Mar. 20, 2025

Related U.S. Application Data
(60) Provisional application No. 63/409,455, filed on Sep. 23, 2022.
(60) Provisional application No. 63/527,795, filed on Jul. 19, 2023.
(60) Provisional application No. 63/515,519, filed on Jul. 25, 2023.

(51) Int. Cl. C12N 15/115; A61K 9/00; A61K 31/711; A61P 31/14; C12Q 1/70

(52) U.S. Cl. CPC C12N 15/115; A61K 9/007; A61K 31/711; A61P 31/14; C12Q 1/70

(57) ABSTRACT

The present disclosure relates to molecular recognition elements (MREs) and, in particular, to aptamers for recognizing and binding to a microorganism, a virus, and/or a molecule present on a microorganism or virus, such as spike protein of severe acute respiratory syndrome coronavirus 2, and methods and uses thereof.

### FIELD OF THE INVENTION

The present disclosure relates to the field of molecular recognition elements (MREs) and, in particular, to aptamers for recognizing and binding to a microorganism, a virus, and/or a molecule present on a microorganism or virus, such as spike protein of severe acute respiratory syndrome coronavirus 2, and methods and uses thereof.

### BACKGROUND OF THE INVENTION

The SARS-CoV-2 (severe acute respiratory syndrome coronavirus 2) virus and COVID-19 pandemic continue to pose a worldwide threat despite vaccination efforts. One of the defining features of the virus is the spike(S) glycoprotein, which decorates the surface of the pathogen and mediates viral entry into the host cell. Although other SARS-CoV-2 proteins can potentially serve as antigen targets, several factors establish the S protein as a particularly useful target. Notably, its function is indispensable to the virus and its protruding nature offers the potential for detection without the need for lysis. Furthermore, the S protein is trimeric, possessing three identical monomers that arrange in a symmetrical shape. This offers the opportunity to engineer compatible trimeric molecular recognition elements (MREs), capable of symmetrically recognizing multiple subunits of the same S protein.

An extensive array of MREs have been investigated for SARS-CoV-2 research since the onset of the pandemic. Amongst these MREs, nucleic acid aptamers have stood out as a promising option. Aptamers are short, single-stranded oligonucleotides that demonstrate high binding affinity and selectivity towards a specific substrate. However, there is currently a need for improved sensitivity and specificity among nucleic acid aptamers for detection of SARS-CoV-2 and other trimeric proteins.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a homotrimeric aptamer for molecular recognition, and methods of making and uses thereof. In accordance with one aspect of the invention, there is provided a homotrimeric DNA aptamer comprising an aptameric nucleotide sequence that binds to a homotrimeric protein in a symmetric configuration, a linker, and a connector molecule. In a further embodiment, there is provided a homotrimeric DNA aptamer comprising three aptameric nucleotide sequences that each bind to a homotrimeric protein in a symmetric configuration, a linker, and a connector molecule. In certain embodiments, the homotrimeric DNA aptamer described herein comprises a connector molecule having three arms, each arm being linked to one of the three aptameric nucleotide sequences by a linker.

In further embodiments, the homotrimeric DNA aptamer described herein comprises aptameric nucleotide sequences that bind to a homotrimeric severe acute respiratory syndrome coronavirus 2 (SARS-CoV-2) spike protein, or a variant thereof. In some embodiments, the homotrimeric DNA aptamer described herein comprises aptameric nucleotide sequences selected from the group consisting of SEQ ID NOS: 1-100, a functional fragment, and a functional variant thereof. In other embodiments, the aptameric nucleotide sequences are selected from the group consisting of SEQ ID NOS: 1-10, a functional fragment, and a functional variant thereof. In further embodiments, the aptameric nucleotide sequences are selected from the group consisting of SEQ ID NOS: 103-108, a functional fragment, and a functional variant thereof. In certain embodiments, the aptameric nucleotide sequences comprise SEQ ID NO: 103 or 108, or a functional fragment and/or functional variant thereof. In other embodiments, the homotrimeric DNA aptamer described herein comprises SEQ ID NO: 109 or 110, or a functional fragment and/or functional variant thereof.

In some embodiments, the trimeric aptamer comprises a linker that is a nucleotide or non-nucleotide linker. In certain embodiments, the linker comprises a natural or a synthetic nucleotide linker. In further embodiments, the linker comprises polythymidine. In other embodiments, the linker comprises 15-thymine.

In some embodiments, the homotrimeric DNA aptamer described herein comprises a connector molecule that is a trebler biomolecule. In certain embodiments, the trebler biomolecule comprises phosphoramidite.

In some embodiments, the homotrimeric DNA aptamer described herein has a symmetric configuration that structurally aligns with the symmetric configuration of the homotrimeric protein. In certain embodiments, the homotrimeric DNA aptamer described herein can universally identify SARS-CoV-2 spike protein and variants thereof. In some embodiments, the homotrimeric DNA aptamer described herein binds the SARS-CoV-2 spike protein with picomolar affinity or femtomolar affinity.

In accordance with another aspect of the invention, there is provided a use of the homotrimeric DNA aptamer described herein for detecting SARS-CoV-2 spike protein and variants thereof. In a further embodiment, there is provided a biosensor comprising the homotrimeric DNA aptamer described herein immobilized on and/or in a material for detecting SARS-CoV-2 spike protein and variants thereof.

In accordance with a further embodiment, there is provided a method for detecting the presence of a SARS-CoV-2 spike protein and variants thereof in a sample, the method comprising:
a) Contacting the sample with the aptamer described herein, wherein the aptamer binds the SARS-CoV-2 spike protein or variants thereof; and
b) Detecting the binding of the aptamer with the SARS-CoV-2 spike protein or variants thereof.

In some embodiments, detecting the binding of the aptamer with the homotrimeric protein comprises detecting a signal including but not limited to a fluorescent, colorimetric, electrochemical, surface plasmon resonance (SPR) or radioactive signal, wherein detecting of a signal indicates the presence of the homotrimeric protein in the sample. In certain embodiments, an enzyme-linked aptamer binding assay with nanozymes is used for colorimetric detection. In further embodiments, the nanozymes comprise Pd—Ir nanocubes. In some embodiments, the method detects SARS-CoV-2 infection in a subject. In some embodiments, the sample comprises, but is not limited to, tissue samples, saliva, sputum, urine, blood, serum, other bodily fluids and/or secretions.

In accordance with another embodiment, there is provided a kit for detecting SARS-CoV-2 spike protein or variants thereof in a sample, wherein the kit comprises the biosensor described herein, or components required for the method described herein, and instructions for use of the kit.

In accordance with another aspect of the invention, there is provided a therapeutic formulation comprising the homotrimeric DNA aptamer described herein, and a solvent, a carrier, a diluent, and/or a dispersant.

In accordance with another aspect of the invention, there is provided a composition comprising a therapeutically effective amount of the homotrimeric DNA aptamer described herein, for treating a SARS-CoV-2 viral infection in a subject. In some embodiments, the composition is for administration into the airway, bronchus or lungs via an intranasal route. In other embodiments, the composition is for administration into the airway, bronchus or lungs via an inhalation route.

In accordance with another aspect, the invention provides a method for treating a viral infection in a subject comprising administering to the subject an effective amount of the composition comprising the homotrimeric DNA aptamer described herein. In certain embodiments, the viral infection is SARS-CoV-2 virus.

In accordance with another aspect of the invention, there is provided a kit comprising a container having contained therein a composition comprising the homotrimeric DNA aptamer described herein, the container adapted to deliver the composition by an intranasal or pulmonary route.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments but should be given the broadest interpretation consistent with the description as a whole.

### BRIEF DESCRIPTION OF THE DRAWINGS

These and other features of the invention will become more apparent in the following detailed description in which reference is made to the appended drawings.

FIG. 1 shows a design of a trimeric aptamer for the trimeric spike protein of SARS-CoV-2 in exemplary embodiments of the disclosure: (A) Top view of the SARS-CoV-2 trimeric spike protein showing the complementarity with the trimeric aptamer to achieve molecular recognition. The trimeric structure of the aptamer ligand complements the molecular scaffold of the spike protein and optimizes binding affinity; (B) Secondary structure of SARS-CoV-2 spike protein binding aptamer MSA52. The ability of MSA52 to universally identify ongoing and predicted SARS-CoV-2 variants of concern provides an ideal candidate for COVID-19 detection; (C) Construction of the trimeric aptamer with MSA52, trebler, and 15-thymine linker. The trebler biomolecule enables the assembly of a symmetric, multimeric recognition element.

FIG. 2 shows a size analysis of TMSA52, DMSA52 and MSA52 using dPAGE in exemplary embodiments of the disclosure. The DNA sequences are listed in Table 4. The mobility of linear monomeric aptamer (MSA52) and linear dimeric aptamer (DMSA52) are consistent with DNA ladder. The mobility of branched trimeric aptamer (TMSA52) is significantly slower than the corresponding DNA ladder due to the branched structure. The result showed that branched trimeric aptamer (TMSA52) was synthesized successfully.

FIG. 3 shows an assessment of the binding affinity of trimeric aptamer in exemplary embodiments of the disclosure: (A) the wild-type (WT), B.1.1.7, B.1.351, and P.1 spike protein variants; (B) the B.1.429, B.1.617.1, B.1.617.2, and B.1.1.529 spike protein variants; (C) SARS-CoV-1 spike protein, and RBD of seasonal coronavirus 229E and OC43 by dot-blot assays; (D) The corresponding plots of bound fraction against protein concentrations in panel (C) show the binding affinity of trimeric aptamer for SARS-CoV-1 spike protein, and RBD of seasonal coronavirus 229E and OC43. BA: bound aptamer; UA: unbound aptamer.

FIG. 4 shows an assessment of binding affinity of TMSA52 in exemplary embodiments of the disclosure: (A) SARS-CoV-2 spike protein variants; (B) pseudotyped lentiviruses displaying the spike protein variants. Kd values displayed consistently high affinity (pM to fM range) for both WT and variant strains.

FIG. 5 shows an assessment of the binding affinity of trimeric aptamer in exemplary embodiments of the disclosure: (A) the pseudotyped lentiviruses expressing the wild-type (WT), B.1.1.7, B.1.351, and P.1 spike protein variants; (B) the pseudotyped lentiviruses expressing the B.1.429, B.1.617.1, B.1.617.2, and B.1.1.529 spike protein variants; (C) control lentiviruses by dot-blot assays; (D) The corresponding plot of bound fraction against virus concentrations in panel (C) shows the binding affinity of trimeric aptamer for control lentiviruses. BA: bound aptamer; UA: unbound aptamer.

FIG. 6 shows validation of the cooperative effect amongst the aptamer arms of TMSA52 for the binding of spike protein in exemplary embodiments of the disclosure: (A) Blocking of trimeric aptamer (TMSA52) arms using various AS: TMSA52 ratios. AS is a 40-nt single-stranded DNA sequence that is complementary to aptamer MSA52; (B) Electrophoretic mobility shift assay; (C) corresponding plot displaying the hybridization efficiency between 32P-AS and TMSA52 at different AS: TMSA52 ratios.

FIG. 7 shows the cooperativity of binding by three aptamer arms in exemplary embodiments of the disclosure: (A) Dot-blot assays; (B) corresponding binding affinity curves of TMSA52 for B.1.1.529 spike protein at different AS: TMSA52 ratios. BA: bound aptamer; UA: unbound aptamer.

FIG. 8 shows an assessment of binding affinity in exemplary embodiments of the disclosure: (A) monomeric aptamer (MSA52); (B) dimeric aptamer (DMSA52) for B.1.1.529 S by dot-blot assays; (C) Plots of bound fraction against B.1.1.529 S concentrations in panels (A) and (B) showing the binding affinity of monomeric aptamer (MSA52) and dimeric aptamer (DMSA52) for B.1.1.529 S. BA: bound aptamer; UA: unbound aptamer.

FIG. 9 shows a design of an enzyme-linked aptamer binding assay for colorimetric detection of SARS-CoV-2 using TMSA52 in exemplary embodiments of the disclosure: (A) Illustration of colorimetric sandwich assay in which SARS-CoV-2 pseudoviruses are captured with biotinylated aptamers, using Pd—Ir nanocubes as peroxidase-mimicking nanozymes; SA: streptavidin: (B) Photograph of colorimetric test; (C) corresponding concentration-response plots using A₄₅₀ for pseudotyped lentivirus expressing the SARS-CoV-2 B.1.1.529 spike protein in buffer, using the biotinylated trimeric aptamer (TMSA52-B) at different AS: TMSA52-B ratios; AS is a 40-nt single-stranded DNA sequence that is complementary to aptamer MSA52; (D) Response based on A₄₅₀ (inset: photograph) demonstrating the specificity of the trimeric aptamer-based method for the detection of B.1.1.529 pseudovirus. SARS-CoV-1 spike protein, spike RBD of seasonal coronaviruses 229E and OC43, human IgG, amylase, BSA, and lentivirus are used as controls. The concentrations of control proteins were 10 nM; the concentrations of pseudovirus and lentivirus were 10⁵ cp/mL (corresponding to 120 aM). LOD: limit of detection, 3 times the standard deviation of blank samples.

FIG. 10 shows TEM characterization of Pd—Ir nanocubes in exemplary embodiments of the disclosure.

FIG. 11 shows a photograph and corresponding plots of absorbance in exemplary embodiments of the disclosure: (A) The photograph: (B) corresponding plots of absorbance at 450 nm showing the detection of the pseudotyped lentiviruses expressing the B.1.1.529 spike protein variant by biotinylated monomeric aptamer (MSA52-B), dimeric aptamer (DMSA52-B), and trimeric aptamer (TMSA52-B). LOD: limit of detection, 3 times the standard deviation of blank samples.

FIG. 12 shows the detection of pseudoviruses spiked human saliva in exemplary embodiments of the disclosure: (A) Photograph of colorimetric test; (B and C) corresponding plots depicting the detection of pseudoviruses expressing different spike protein variants spiked in pooled human saliva using the ELABA assay with TMSA52. LOD: limit of detection, 3 times the standard deviation of blank samples.

FIG. 13 shows clinical evaluation of the trimeric aptamer assay in exemplary embodiments of the disclosure: (A) Schematic illustration; (B) signal response of the TMSA52-based ELABA for the detection SARS-CoV-2 in clinical saliva samples, including 50 NPS positive samples (wild-type SARS-CoV-2, Alpha (B.1.1.7), Gamma (P.1), Delta (B.1.617.2), Omicron (B.1.1.529: PS9-PS21, BA.2: PS22-PS23, BA.4: PS24-PS26, BA.5: PS27-PS36, BA.2.12.1: PS37-PS44) and undetermined variants) and 60 NPS negative samples. PS: positive saliva sample; NS: negative saliva sample; PC: positive control, 4×10⁴ cp/mL B.1.1.529 pseudovirus spiked in commercial pooled human saliva; NC: negative control, commercial pooled human saliva. The dotted line marks the cut-off point for the TMSA52-based assay. The error bars stand for the standard deviation of three (n=3) replicated samples. ND: not detected. NPS: nasopharyngeal swab.

FIG. 14 shows a Receiver Operating Characteristic (ROC) curve to determine the clinical cut-off absorbance value in exemplary embodiments of the disclosure: (A) Box and whisker plot depicting the distribution of the NPS positive and negative patient saliva samples presented in FIG. 12B; (B) Receiver-Operator Characteristics Curve for the TMSA52-based ELABA. The overall accuracy (AUC: Area Under the Curve) was 0.929 (95% confidence interval: 0.875-0.983) with an optimum sensitivity of 84.0% (true positive cases detected) and a corresponding specificity of 98.3% (true negative cases detected) at a threshold absorbance (450 nm) of 0.027.

FIG. 15 shows BTNX COVID-19 antigen rapid test for the detection SARS-CoV-2 in clinical saliva samples in exemplary embodiments of the disclosure. Including 50 NPS positive samples and 60 NPS negative samples (grey). PS: Positive saliva sample; NS: Negative saliva sample. C: control line; T: test line; NPS: nasopharyngeal swab; (+) positive; (−) negative.

FIG. 16 shows percent neutralization of SARS-CoV-2 Omicron BA.1 (A) or MA10 (B) by aptamer constructs or S309 antibody in vitro on Vero E6 cells at 3 days post-infection to assess the aptamers ability to neutralize SARS-CoV-2 in vitro. Briefly, serial dilutions of TMSA52 (1 μM), Ir-TMSA52 (1 μM), Ir (1 μM), and S309 (1 μg/mL) were incubated with the virus, and the mixture was plated on Vero E6 cells for 3 days prior to measuring the cell viability using Cell Titre Glo 2.0. Open circles represent TMSA52 (universal SARS-CoV-2 aptamer), closed circles represent Ir-TMSA52 (universal SARS-CoV-2 aptamer on an iridium nanoplate), open triangles represent Ir (Iridium nanoplate), closed triangles represent S309 (SARS-CoV-2 monoclonal neutralizing antibody).

FIG. 17 shows the in vivo assessment of aptamers in protection against non-lethal challenge with SARS-CoV-2. (A) illustrates the experimental schema. Animals were treated intranasally (i.n.) with either nuclease free water, TMSA52 (universal SARS-CoV-2 aptamer), Ir-TMSA52 (universal SARS-CoV-2 aptamer on an iridium nanoplate), Ir (iridium nanoplate), S309 (SARS-CoV-2 monoclonal neutralizing antibody), or mTMSA52 (a mutated TMSA52 with partially abrogated binding to SARS-CoV-2 spike) 2 hours prior to challenge with a sub-lethal dose (10⁴ PFU) of SARS-CoV-2 MA10. Animals were subsequently monitored for weight loss and mortality for 7 days. A cohort of animals (n=5) was sacrificed 4 days post-infection for determination of lung viral burden. (B) shows the weight loss following treatment, in comparison to nuclease-free water treated controls. (C) shows the viral burden (Log₁₀ TCID₅₀) in the lung at 4 days post-SARS-CoV-2 infection. Briefly, lungs were homogenized, and plated on Vero E6 cells for 5 days before enumeration by visually assessing cytopathic effect. Closed squares represent control animals, open circles represent TMSA52, closed circles represent Ir-TMSA52, open triangles represent Ir, closed triangles represent S309, open squares represent mutant aptamer. Data is represented mean±SEM. Statistical analysis for panel E was a one-way ANOVA with Tukey multiple comparisons test. Data is representative of 1 independent experiment, ns=not significant.

FIG. 18 shows the in vivo assessment of aptamers in protection against lethal challenge with SARS-CoV-2. (A) illustrates the experimental schema. Animals were treated intranasally (i.n.) with either nuclease free water, TMSA52 (universal SARS-CoV-2 aptamer), Ir-TMSA52 (universal SARS-CoV-2 aptamer on an iridium nanoplate), Ir (iridium nanoplate alone), S309 (SARS-CoV-2 monoclonal neutralizing antibody), or scrambled aptamer (a scrambled TMSA52 with equimolar A:T and C:G ratios with abrogated binding to SARS-CoV-2 spike) 2 hours prior to challenge with a lethal dose (10⁵ PFU) of SARS-CoV-2 MA10. Animals were subsequently monitored for weight loss and mortality for 7 days. A cohort of animals (n=5) was sacrificed 4 days post-infection for determination of lung viral burden and histopathology. (B) shows the weight loss following treatment, in comparison to nuclease-free water treated controls. (C) shows the Kaplan-Meier survival curve depicting percent-survival over the course of infection. Closed squares represent control animals, open circles represent TMSA52, closed circles represent Ir-TMSA52, open triangles represent Ir, closed triangle represent S309, open squares represent scrambled aptamer. (D) shows the clinical scoring. The number indicates the total number of clinical manifestations (fur ruffling, rapid respiration, lethargy, and/or hunched posture). (E) shows the gross histopathological representative images of lungs collected 4 days post-SARS-CoV-2 infection. Arrows indicate areas of diffuse hemorrhage. (F) shows the viral burden (Log₁₀ TCID₅₀) in the lung at 4 days post-SARS-CoV-2 infection. Briefly, lungs were homogenized, and plated on Vero E6 cells for 5 days before enumeration by visually assessing cytopathic effect. Closed squares represent control animals, open circles represent TMSA52, closed circles represent Ir-TMSA52, open triangles represent Ir, closed triangle represent S309, open squares represent scrambled aptamer. Data is represented mean±SEM. Statistical analysis for panel F was a one-way ANOVA with Tukey multiple comparisons test. Data is representative of 1 independent experiment, ns=not significant.

### DETAILED DESCRIPTION OF THE INVENTION

Aptamer binding affinity can be further enhanced by creating multimeric aptamers by joining two or more monomeric substituents together. The adaptation of symmetric, multimeric proteins to selectively bind DNA sequences is a naturally occurring phenomenon in biological systems.¹ Their interdependent, molecular scaffolds create one cohesive binding site for the recognition of a DNA substrate. Type II restriction enzymes, such as EcoRV, are just one of many examples of dimeric proteins that rely on two identical domains to recognize palindromic, double-stranded DNA.² A simple point mutation is enough to significantly hinder enzymatic activity and disrupt this dimer-dimer interaction. Biological activity is ultimately improved by this compatible alignment between a symmetric multimeric protein and a symmetric multimeric target.

Whilst the example of EcoRV is one that naturally occurs, the inventors of the instant invention harnessed the idea of a symmetrical recognition mechanism and innovatively adapted this idea into a synthetic system. In this regard, the present invention provides a homotrimeric aptamer for use as molecular recognition elements (MREs) that can be used for bioanalytical and therapeutic applications. According to embodiments, the homotrimeric aptamer comprises aptameric nucleotide sequences that cooperatively bind to a homotrimeric protein in a symmetric configuration. In certain embodiments, the homotrimeric aptamer comprises homomeric aptameric nucleotide sequences arranged in a trimeric structure for the purpose of increasing target binding affinity via the arrangement of the homomeric aptameric nucleotide sequences via a connector molecule to bind to a homotrimeric protein in a symmetric configuration.

In certain embodiments, the homotrimeric DNA aptamer comprises aptameric nucleotide sequences that together form a symmetric interaction between the homotrimeric DNA aptamer and the homotrimeric protein. In preferred embodiments, the homotrimeric DNA aptamer comprises a connector molecule having three arms, e.g., a trebler biomolecule, wherein each arm is linked to an aptameric nucleotide sequence by a linker. In some embodiments, the aptameric nucleotide sequence on each arm of the connector molecule is identical. According to embodiments, each aptameric nucleotide sequence is an aptamer that binds to homotrimeric severe acute respiratory syndrome coronavirus 2 (SARS-CoV-2) spike protein, or a variant thereof. When configured in a symmetric trimeric formation, the three aptameric nucleotide sequences on the respective arms of the connector associate with the three spike protein subunits of the homotrimeric SARS-CoV-2 spike protein in concerted fashion.

In some embodiments, the homotrimeric DNA aptamer described herein has a symmetric configuration that structurally aligns with the symmetric configuration of the homotrimeric protein. In certain embodiments, the homotrimeric DNA aptamer described herein can universally identify SARS-CoV-2 spike protein and variants thereof. In some embodiments, the homotrimeric DNA aptamer described herein binds the SARS-CoV-2 spike protein with picomolar affinity or femtomolar affinity.

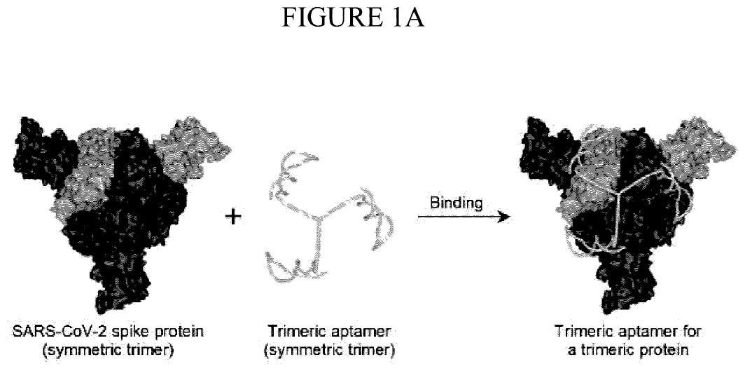

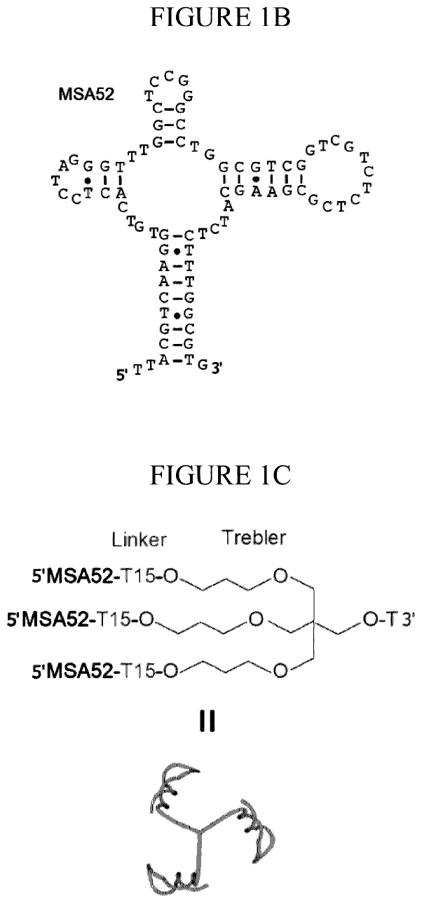

According to embodiments of the present invention, the symmetric homotrimeric DNA aptamer comprises aptameric nucleotide sequences selected from the MSA52 aptamer family (Table 1) which has been identified as displaying universally high affinity for the spike proteins of SARS-CoV-2 as well as variants including the Alpha, Beta, Gamma, Epsilon, Kappa Delta, and Omicron variants. In further embodiments, the homotrimeric DNA aptamer comprises aptameric nucleotide sequences corresponding to the anti-S protein DNA aptamer (hereinafter referred to as “TMSA52”). Such embodiments provide for precision molecular recognition of the symmetric homotrimeric spike protein of severe acute respiratory syndrome coronavirus 2 (SARS-CoV-2). In particular, the configuration of TMSA52, according to embodiments described herein, complements and structurally aligns with the trimeric nature of the S protein (FIG. 1A). In such embodiments, the TMSA52 binds several SARS-CoV-2 spike protein variants with picomolar affinity and diverse pseudotyped lentiviruses expressing SARS-CoV-2 spike protein variants with femtomolar affinity.

According to further embodiments, the invention provides for enhanced binding specificity and affinity to the symmetric homotrimeric SARS-CoV-2 spike protein and variants thereof. According to such embodiments, the trimeric form of the homomeric (MSA52) aptamers enhance the binding interaction by simultaneously binding the spike protein subunits. In some embodiments, the symmetric homotrimeric DNA aptamer (e.g., TMSA52) synergistically enhances the binding interaction with symmetric homotrimeric SARS-CoV-2 spike protein, and variants thereof, by more than two orders of magnitude over MSA52. In certain embodiments, the symmetric homotrimeric DNA aptamer (e.g., TMSA52) synergistically enhances the binding interaction with symmetric homotrimeric SARS-CoV-2 spike protein and variants thereof.

Some embodiments of the invention provide for use of the homotrimeric DNA aptamer in diagnostics or bioanalytical applications. For example, the homotrimeric DNA aptamer may be utilized in a biosensor. In certain embodiments, the TMSA52 aptamer is used in conjunction with Pd—Ir nanocube (a nanozyme). According to such embodiments, the biosensor was found capable to sensitively detect diverse pseudotyped lentiviruses in pooled human saliva with a limit of detection as low as 6.3×10³ cp/mL, for example. In further embodiments, when used to test 110 COVID-19 positive and negative saliva samples, the biosensor of the invention was found to provide sensitivity and specificity values of 84.0% and 98.3%, respectively. Accordingly, embodiments of the invention provide homotrimeric DNA aptamers, e.g., TMSA52, for use in rapid tests for COVID-19.

According to further embodiments, the present invention provides a multipurpose attachment site on the 3′ branch/arm of the trebler connector. In such embodiments, the 3′ arm may be functionalized with a variety of other molecules to enable i) surface immobilization or crosslinking via biotin, amine, thiol or digoxigenin molecules, ii) attachment of reporter elements such as fluorophores, enzymes (HRP), nucleic acid amplification primers (RCA, PCR, LAMP, etc.) or iii) drug delivery such as small molecule drugs, antimicrobial agents, and gene therapeutics.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments but should be given the broadest interpretation consistent with the description as a whole.

### Definitions

Unless otherwise indicated, the definitions and embodiments described in this and other sections are intended to be applicable to all embodiments and aspects of the present disclosure herein described for which they are suitable as would be understood by a person skilled in the art. It is also to be understood that the terminology used herein is for the purpose of describing particular aspects only and is not intended to be limiting.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies. In addition, all ranges given herein include the end of the ranges and also any intermediate range points, whether explicitly stated or not.

As used in this disclosure, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The abbreviation, “e.g.” is derived from the Latin exempli gratia and is used herein to indicate a non-limiting example. Thus, the abbreviation “e.g.” is synonymous with the term “for example.” The word “or” is intended to include “and” unless the context clearly indicates otherwise.

The term “sample” or “test sample” as used herein refers to any material in which the presence or amount of a target analyte is unknown and can be determined in an assay. The sample can be from any source, for example, any biological (e.g. human or animal samples, including clinical samples), environmental (e.g. water, soil or air) or natural (e.g. plants) source, or from any manufactured or synthetic source (e.g. food or drinks). The sample can be comprised or is suspected of comprising one or more analytes. The sample can be a “biological sample” comprising cellular and non-cellular material, including, but not limited to, tissue samples, saliva, sputum, urine, blood, serum, other bodily fluids and/or secretions. In some embodiments, the sample comprises saliva, sputum, oropharyngeal and/or nasopharyngeal secretions. In some embodiments, the sample comprises saliva.

The term “target”, “analyte” or “target analyte” as used herein refer to any agent, including, but not limited to, a small inorganic molecule, small organic molecule, metal ion, biomolecule, toxin, biopolymer (such as a nucleic acid, carbohydrate, lipid, peptide, protein), cell, tissue, microorganism and virus, for which one would like to sense or detect. The analyte can be either isolated from a natural source or synthetic. The analyte can be a single compound or a class of compounds, such as a class of compounds that share structural or functional features. The term analyte also includes combinations (e.g. mixtures) of compounds or agents such as, but not limited, to combinatorial libraries and samples from an organism or a natural environment.

The term “subject” as used herein includes all members of the animal kingdom including mammals such as a mouse, a rat, a dog and a human.

The term “virus” as used herein refer to an organism of simple structure, composed of proteins and nucleic acids, and capable of reproducing only within specific living cells, using its metabolism. In some embodiments, the virus is an enveloped virus, a non-enveloped virus, a DNA virus, a single-stranded RNA virus and/or a double-stranded RNA virus. Non-limiting examples of virus include rhinovirus, myxovirus (including influenza virus), paramyxovirus, coronavirus such as SARS-CoV-2, norovirus, rotavirus, herpes simplex virus, pox virus (including variola virus), reovirus, adenovirus, enterovirus, encephalomyocarditis virus, cytomegalovirus, varicella zoster virus, rabies lyssavirus and retrovirus (including HIV).

The term “severe acute respiratory syndrome coronavirus 2”, “coronavirus 2”, or “SARS-CoV-2” as used herein refer to a coronavirus first identified in Wuhan, China in 2019 that causes coronavirus disease (COVID-19). The term includes any variant of the SARS-CoV-2 virus with a variant and/or mutated nucleic acid sequence from the original version identified in Wuhan. Variants includes, but are not limited to, UK B.1.1.7 (501Y.V1), South Africa B.1.351 (501Y.V2), Brazil P.1 (501Y.V3) and India B.1.617.

The term “nucleic acid” as used herein refers to a biopolymer comprising monomers of nucleotides, such as deoxyribonucleic acid (DNA), ribonucleic acid (RNA) and other polynucleotides of modified nucleotides and/or nucleotide derivatives, and can be either double stranded (ds) or single stranded (ss). In some embodiments, modified nucleotides contain one or more modified bases (e.g. unusual bases such as inosine, and functional modifications to the bases such as amino), modified backbones (e.g. peptide nucleic acid, PNA) and/or other chemically, enzymatically, or metabolically modified forms.

The term “aptamer” as used herein refers to a short, chemically synthesized nucleic acid molecule or oligonucleotide sequence which can be generated by in vitro selection to fold into specific three-dimensional structures that bind to a specific analyte with dissociation constants, for example, in the pico- to nano-molar range. Aptamers can be single-stranded DNA, RNA, modified nucleotides and/or nucleotide derivatives. Aptamers can also be naturally occurring RNA aptamers termed “riboswitches”. Functional aptamer sequences can also be rationally designed, truncated, conjugated or otherwise modified from original parent (or full length) sequences. A functional fragment of an aptamer is the portion of an aptamer that retains aptameric function, for example, function in binding to molecules such as protein, lipid, carbohydrate, and nucleic acid. A functional variant of an aptamer refers to an aptamer that has been modified, with nucleotide derivates or otherwise, elongated or truncated, and still retains aptameric function.

The term “hybridizes”, “hybridized” or “hybridization” as used herein refers to the sequence specific non-covalent binding interaction with a complementary, or partially complementary, nucleic acid sequence. When, for example, the 5′-end region of an aptamer hybridizes to the 3′-end region, it can form a duplex DNA element.

The term “biosensor” as used herein refers to a device that incorporates a biological entity as a molecular recognition element and is capable of producing a measurable signal upon binding of a target analyte to the molecular recognition element. The biosensor can also be part of a larger device.

The terms “attenuate”, “inhibit”, “prevent”, “treat”, and grammatical variations thereof, as used herein, refer to a measurable decrease in a given parameter or event.

The term “treatment method”, or “method for the treatment of a pathology or disorder”, means therapy aimed at restoring the health condition of a subject, maintaining the existing condition and/or preventing the worsening of said health condition.

It will be understood that any component defined herein as being included may be explicitly excluded by way of proviso or negative limitation, such as any specific compounds or method steps, whether implicitly or explicitly defined herein.

### Homotrimeric Aptamers

Homotrimeric aptamers are provided that comprise the arrangement of aptameric nucleotide sequences in a trimeric structure. According to embodiments, the homotrimeric aptamer construct comprises a central connector molecule having 3 variable arms to which each aptameric nucleotide sequence is attached via a linker.

According to certain embodiments, the central connector molecule can be a trebler to form the symmetrical trimeric construct. In some embodiments the homotrimeric aptamer is a symmetric trebler construct comprising three identical aptameric nucleotide sequences and linkers attached to the three variable arms of the connector.

According to further embodiments, the connector molecule comprises a multipurpose attachment site on the 3′ arm of the connector, e.g., trebler. In such embodiments, the 3′ arm may be functionalized with a variety of other molecules to enable i) surface immobilization or crosslinking via biotin, amine, thiol or digoxigenin molecules, for example ii) attachment of reporter elements such as fluorophores, enzymes (HRP), nucleic acid amplification primers (RCA, PCR, LAMP, etc.) or iii) drug delivery such as small molecule drugs, antimicrobial agents, and gene therapeutics. In certain embodiments, the 3′ arm comprises another aptameric nucleotide sequence that may be identical or different to the three aptameric nucleotide sequences forming the trimeric construct. In certain embodiments, for example for reporter constructs, the 3′ arm of the connector may be functionalized with a fluorescent reporter molecule, antigen, or enzyme. In other embodiments, for example for immobilization constructs, the 3′ arm of the connector may be functionalized to enable crosslinking, for example with thiol, amide, biotin, digoxigenin, azide, alkyne, carboxyl, or crosslinkers compatible with Click-Chemistry methodologies.

### Aptameric Nucleotide Sequences

According to embodiments, the aptameric nucleotide sequences are aptamers specific for one or more epitopes of a homotrimeric protein in a symmetric configuration. According to embodiments, the aptameric nucleotide sequences are aptamers specific for a homotrimeric severe acute respiratory syndrome coronavirus 2 (SARS-CoV-2) spike protein or a variant thereof. A variety of aptameric nucleotide sequences that are specific to the homotrimeric severe acute respiratory syndrome coronavirus 2 (SARS-CoV-2) spike protein, or a variant thereof, are known in the art. Appropriate aptameric nucleotide sequences can be readily selected by one skilled in the art.

In one embodiment, the homotrimeric aptamer comprises aptameric nucleotide sequences specific for the homotrimeric SARS-CoV-2 spike protein. In other embodiments, the homotrimeric aptamer comprises aptameric nucleotide sequences specific for one or more variants of the homotrimeric SARS-CoV-2 spike protein. In further embodiments, the homotrimeric aptamer comprises aptameric nucleotide sequences specific for the homotrimeric SARS-CoV-2 spike protein and variants thereof.

According to certain embodiments, the homotrimeric aptamer comprises aptameric nucleotide sequences from the MSA52 aptamer family. There are a total of 321 members of the MSA52 aptamer family (International Patent Application No. PCT/CA2023/050029). The top 100 members of the MSA52 aptamer family were identified by the inventors as exhibiting the highest binding specificity to spike proteins of the wildtype SARSCoV-2 and its eight current variants of concern, including B.1.1.7, B.1.351, P.1, B.1.429, B.1.617.1 B.1.617.2, B.1.617.2.1 and B.1.1.529, while still retaining selectivity against non-SARS-CoV-2 spike proteins. Table 1 lists the top 100 members of the MSA52 aptamer family.

TABLE 1
MSA52 Aptameric Nucleotide Sequences

| SEQ | |
|---|---|
| ID NO. | SEQUENCE |
| 1 | GTAGGGTTTGGCTCCGGGCCTGGCGTCGGTCGTCTCTCGC |
| 2 | -TAGGGTTTGGCTCCGGGCCTGGCGTCGGTCGTCTCTCGC |
| 3 | ATAGGGTTTGGCTCCGGGCCTGGCGTCGGTCGTCTCTCGC |
| 4 | GTAGGGTTTGGCTCCGGGCCTGGCGTCGGTCGTCTCTCGT |
| 5 | TTAGGGTTTGGCTCCGGGCCTGGCGTCGGTCGTCTCTCGC |
| 6 | GTAGGGTTTGGCTCCGGGCCTGGCGTCGGTCGTCTTTCGC |
| 7 | GTAGGGTTTGGCTCCGGGCCTGGCGTCGGTCGTCTCTTGC |
| 8 | G-CGGGTTTGGCTCCGGGCCTGGCGTCGGTCGTCTCTCGC |
| 9 | GTAGGGTTTGGCTCCGGGCCTGGCGTCGGTCGTCTCCCGC |
| 10 | GCAGGGTTTGGCTCCGGGCCTGGCGTCGGTCGTCTCTCGC |
| 11 | CTAGGGTTTGGCTCCGGGCCTGGCGTCGGTCGTCTCTCGC |
| 12 | GTAGGGTTTGGCTCCGGGCCTGGCGTCGGTTGTCTCTCGC |
| 13 | GTAGGGTTTGGCTCCGGGCCTGGCGTCGGTCATCTCTCGC |
| 14 | GTAGGGTTTGGCTCCGGGCCTGGCGTCGGTCGTCCCTCGC |
| 15 | GTGGGGTTTGGCTCCGGGCCTGGCGTCGGTCGTCTCTCGC |
| 16 | GTAGGGTTTGGCTCCGGGCCTGGCGTAGGTCGTCTCTCGC |
| 17 | GAAGGGTTTGGCTCCGGGCCTGGCGTCGGTCGTCTCTCGC |
| 18 | GTAGGGTTTGGCTCCGGGCCTGGCGTCGGTCGCCTCTCGC |
| 19 | GTAGGGTTTGGCTCCGGGCCTGGCGTCGGTCGTCTCTCGA |
| 20 | AC-GGGTTTGGCTCCGGGCCTGGCGTCGGTCGTCTCTCGC |
| 21 | GTAGGGTTTGGCTCCGGGCCTGGCGTCGGTCGTCTCTCTC |
| 22 | GTAGGGTTTGGCTCCGGGCCTGGCGTCGGTCGTTTCTCGC |
| 23 | ATAGGGTTTGGCTCCGGGCCTGGCGTCGGTCGTCTCTCGT |
| 24 | -T-GGGTTTGGCTCCGGGCCTGGCGTCGGTCGTCTCTCGC |
| 25 | GTAGGGTTTGGCTCAGGGCCTGGCGTCGGTCGTCTCTCGC |
| 26 | GTAGGGTTTGGCTCCGGGCCTGGCGTCGGTCGTCTC-CTC |
| 27 | GTAGGGTTTGGCTCCGGGCCTGGCGTCGGTCCTCTCTCGC |
| 28 | GTAGGGTTTGGCTCCGGGCCTGGCGACGGTCGTCTCTCGC |
| 29 | GTAGGGTTTGGCTCCGGGCCTGGCGTCGGTCTTCTCTCGC |
| 30 | -CAGGGTTTGGCTCCGGGCCTGGCGTCGGTCGTCTCTCGC |
| 31 | --AGGGTTTGGCTCCGGGCCTGGCGTCGGTCGTCTCTCGC |
| 32 | GTAGGGTTTGGCTCCGGACCTGGCGTCGGTCGTCTCTCGC |
| 33 | GTAGGGTTTGGCTCCGGGCCT-GCGTCGGTCGTCTCTCGC |
| 34 | GTAGGGTTTGGCTCCGGGCCTGGCGTCGGTCGTCTCTCAC |
| 35 | GTAGGGTTTGGCTCCGGGCCTGGCGTCGGTCGTCTCTCGG |
| 36 | ACTGGGTTTGGCTCCGGGCCTGGCGTCGGTCGTCTCTCGC |
| 37 | GTAGGGTTTGGCTCCGGGCCTGGCGTCGGTCGTCTCTAGC |
| 38 | GTAGGGCTTGGCTCCGGGCCTGGCGTCGGTCGTCTCTCGC |
| 39 | GTAGGGTTTGGCTCCGGGCCTGGCGTCGGTCGTCTCTCCC |
| 40 | ATGGGGTTTGGCTCCGGGCCTGGCGTCGGTCGTCTCTCGC |
| 41 | GTAGGGTTTGGCTCCGGGCCTGGCGTCGTTCGTCTCTCGC |
| 42 | GTAGGGTTTGGCTCCGGGCCTAGCGTCGGTCGTCTCTCGC |
| 43 | GTAGGGTTTGGCTCCGGGCCTGGCGTCGGTAGTCTCTCGC |
| 44 | GGAGGGTTTGGCATCGGGCCTGGCGTCGGTCGTCTCTCGC |
| 45 | GTAGGGTTTGGCTCCGGGCCTGGCTTCGGTCGTCTCTCGC |
| 46 | GTAGGGTTTGGCTCCGGGCCTGGCGCCGGTCGTCTCTCGC |
| 47 | GTAGGGTTTGGCTCCGGGCCCGGCGTCGGTCGTCTCTCGC |
| 48 | -TGGGGTTTGGCTCCGGGCCTGGCGTCGGTCGTCTCTCGC |
| 49 | ACAGGGTTTGGCTCCGGGCCTGGCGTCGGTCGTCTCTCGC |
| 50 | GTAGGGTCTGGCTCCGGGCCTGGCGTCGGTCGTCTCTCGC |
| 51 | GTAGGGTTTGGCTCCGGGCCTGGCGTCGATCGTCTCTCGC |
| 52 | GTAGGGTTTGGCTCCGGGCCTGGCGTCGGTCGTCTCCCGT |
| 53 | ATAGGGTTTGGCTCCGGGCCTGGCGTCGGTCGTTTCTCGC |
| 54 | ATAGGGTTTGGCTCCGGGCCTGGCGTCGGTCTTCTCTCGC |
| 55 | GTAGGGTTTGGCTACGGGCCTGGCGTCGGTCGTCTCTCGC |
| 56 | GTAGGGTTTGGCTCCGGGCCTGGCGTCGGTCGTGTCTCGC |
| 57 | -TAGGGTTTGGCTCCGGGCCTGGCGTCGTTCGTCTCTCGC |
| 58 | -TAGGGTTTGGCTCCGGGCCTGGCGTCGGTCGTCTCTCGT |
| 59 | TCGGGGGGGTTTGGCTCCGGGCCTGGCGTCGGTCGTCTCTCGC |
| 60 | -TAGGGTTTGGCTCCGGGCCTGGCGTCGGTCGTCTTTCGC |
| 61 | GTAGGGTTTGGCTCCGGGCCTGGCGTCGGCCGTCTCTCGC |
| 62 | GTTGGGTTTGGCTCCGGGCCTGGCGTCGGTCGTCTCTCGC |
| 63 | TCCGTAGGGTTTGGCTCCGGGCCTGGCGTCGGTCGTCTCTCGC |
| 64 | GTAGGGTTTGGCCCCGGGCCTGGCGTCGGTCGTCTCTCGC |
| 65 | GTAGGGTTTGGCTCCAGGCCTGGCGTCGGTCGTCTCTCGC |
| 66 | GTAGGGTTCGGCTCCGGGCCTGGCGTCGGTCGTCTCTCGC |
| 67 | GTAGGGTTTGGCTCCGGGCCTGGCGTCGGTCGTCTCTC -- |
| 68 | -TCGGGTTTGGCTCCGGGCCTGGCGTCGGTCGTCTCTCGC |
| 69 | GTAGGGTTTGGCTCCGGGCCTGGCGTCGGACGTCTCTCGC |
| 70 | GTAGGGTTTGGCTCCGGGCCTGGCGTTGGTCGTCTCTCGC |
| 71 | GTAGGGTTTGGCTCCGGGCCTGGCGTCGGTCGTCTATCGC |
| 72 | ATAGGGTTTGGCTCCGGGCCTGGCGTCGGTTGTCTCTCGC |
| 73 | G-CGGGTTTGGCTCCGGGCCTGGCGTCGGTCGTCTCTCGT |
| 74 | GTAGGGTTTGGCTCCGGGCCTGGCGTCAGTCGTCTCTCGC |
| 75 | GTAGGGTTTGGCTTCGGGCCTGGCGTCGGTCGTCTCTCGC |
| 76 | GTAGGGTTTGGCTCCGGGCCTGGCGTCGGTCGTCGCTCGC |
| 77 | GTAGGGTTTGGCTCCGGGCCTGGCGTCGGTCGACTCTCGC |
| 78 | GTAGGGTTTGGCTCCGGGCCTGGCGTCGGTCGTCACTCGC |
| 79 | GTAGGGTTTGGCTCCGGGCCTGGCGTCTGTCGTCTCTCGC |
| 80 | ATCGGGTTTGGCTCCGGGCCTGGCGTCGGTCGTCTCTCGC |
| 81 | GTAGGGTTTGGCTCCGGGCCTGGCGTCGGTCGTCTC-CCC |
| 82 | -TAGGGTTTGGCTCCGGGCCTGGCGTCGGTCGTCTCTTGC |
| 83 | TTAGGGTTTGGCTCCGGGCCTGGCGTCGGTCGTCTCTCGT |
| 84 | GTAGGGTTTGGCTCTGGGCCTGGCGTCGGTCGTCTCTCGC |
| 85 | G--GGGTTTGGCTCCGGGCCTGGCGTCGGTCGTCTCTCGC |
| 86 | AT-GGGTTTGGCTCCGGGCCTGGCGTCGGTCGTCTCTCGC |
| 87 | GTAGGGTTTGGCTCCGGGCCTGACGTCGGTCGTCTCTCGC |
| 88 | -T-GGGATTGGCTCCGGGCCTGGCGTCGGTCGTCTCTCGC |
| 89 | GTAGGGTTTGGCTCCGGGTCTGGCGTCGGTCGTCTCTCGC |
| 90 | ATAGGGTTTGGCTCCGGGCCTGGCGTCGGTCATCTCTCGC |
| 91 | CACTGGGTTTGGCTCCGGGCCTGGCGTCGGTCGTCTCTCGC |
| 92 | GCAGGGTTTGGCTCCGGGCCTGGCGTCGGTCGTCTCTCGT |
| 93 | G--GGGATTGGCTCCGGGCCTGGCGTCGGTCGTCTCTCGC |
| 94 | GTAGGGTTTGGCTCCGGGACTGGCGTCGGTCGTCTCTCGC |
| 95 | GTAGGGTTTGGCTCCGGGCCTGGCGTCGGTCGTCTCCCCC |
| 96 | -TAGGGTTTGGCTCCGGGCCTGGCGTCGGCCGTCTCTCGC |
| 97 | -TAGGGTTTGGCTCCGGGCCTGGCGTCGGTCATCTCTCGC |
| 98 | GTAGGGTTTGGCTCCGGGCCTGGTGTCGGTCGTCTCTCGC |
| 99 | GTAGGGTTTGGCTCCTGGCCTGGCGTCGGTCGTCTCTCGC |
| 100 | GTAGGGATTGGCTCCGGGCCTGGCGTCGGTCGTCTCTCGC |

Each sequence contains primer regions of TTACGTCAAGGTGTCACTCC (SEQ ID NO: 101) and GAAGCATCTCTTTGGCGTG (SEQ ID NO: 102) at the 5' end and 3' end, respectively. Each point mutation in relation to the top ranked sequence (SEQ ID NO: 1) is in bold.

According to embodiments, the homotrimeric aptamer comprises aptameric nucleotide sequences from the MSA52 aptamer family. In other embodiments, the homotrimeric aptamer comprises aptameric nucleotide sequences selected from the group consisting of SEQ ID NOS: 1-100, a functional fragment, and a functional variant thereof. In other embodiments, the homotrimeric aptamer comprises aptameric nucleotide sequences selected from the group consisting of SEQ ID NOS: 1-10, a functional fragment, and a functional variant thereof.

Table 2 lists exemplary MSA52 aptamers that have been identified by the inventors as exhibiting universal binding recognition for SARSCoV-2 spike protein and at least 7 spike protein variants of SARSCoV-2.

TABLE 2
MSA52 Aptamers

| SEQ | | | |
|---|---|---|---|
| ID | | SIZE | |
| NO | NAME | (nt) | SEQUENCE |
| 103 | MSA52 | 79 | TTACGTCAAG GTGTCACTCC |
| | | | GTAGGGTTTG GCTCCGGGCC |
| | | | TGGCGTCGGT CGTCTCTCGC |
| | | | GAAGCATCTC TTTGGCGTG |
| 104 | MSA52-T1 | 68 | TTACGTCAAG GTGTCATTG |
| | | | GCTCCGGGCC TGGCGTCGGT |
| | | | CGTCTCTCGC GAAGCATCTC |
| | | | TTTGGCGTG |
| 105 | MSA52-T2 | 70 | TTACGTCAAG GTGTCACTCC |
| | | | GTAGGGTTTG CTGGCGTCGG |
| | | | TCGTCTCTCG CGAAGCATCT |
| | | | CTTTGGCGTG |
| 106 | MSA52-T3 | 58 | TTACGTCAAG GTGTCACTCC |
| | | | GTAGGGTTTG GCTCCGGGCC |
| | | | TGGCATCTCT TTGGCGTG |
| 107 | MSA52-T4 | 72 | TTACGTCAAG GTGTCACTCC |
| | | | GTAGGGTTTG GCTCCGGGCC |
| | | | TGGCGTCGGT CGCGAAGCAT |
| | | | CTCTTTGGCG TG |
| 108 | B-MSA52 | 84 | Bio-TTTTTTTACG TCAAGGTGTC |
| | | | ACTCCGTAGG GTTTGGCTCC |
| | | | GGGCCTGGCG TCGGTCGTCT |
| | | | CTCGCGAAGC ATCTCTTTGG CGTG |

According to embodiments, the homotrimeric aptamer comprises aptameric nucleotide sequences selected from the group consisting of SEQ ID NOS: 103-108, a functional fragment, and a functional variant thereof.

Table 3 lists exemplary homotrimeric aptamers comprising MSA52 aptameric nucleotide sequences according to embodiments of the invention.

TABLE 3
TMSA52 Homotrimeric Aptamers

| SEQ | | | |
|---|---|---|---|
| ID | | SIZE | |
| NO | NAME | (nt) | SEQUENCE* |
| 109 | TMSA52 | 282 | TTACGTCAAG GTGTCACTCC |
| | | | GTAGGGTTTG GCTCCGGGCC |
| | | | TGGCGTCGGT CGTCTCTCGC |
| | | | GAAGCATCTC TTTGGCGTG |
| | | | TTTTTTTTTT TTTTT- |
| | | | Trebler |
| 110 | TMSA52-B | 287 | TTACGTCAAG GTGTCACTCC |
| | | | GTAGGGTTTG GCTCCGGGCC |
| | | | TGGCGTCGGT CGTCTCTCGC |
| | | | GAAGCATCTC TTTGGCGTG |
| | | | TTTTTTTTTT TTTTT- |
| | | | Trebler-TTTTT-Biotin |

*sequence for one arm of the trebler connecter molecule.

According to further embodiments, the homotrimeric aptamer comprises SEQ ID NOS: 109 or 110. In other embodiments, the homotrimeric aptamer comprises a connector molecule wherein each arm of the connector is linked to SEQ ID NO: 109 or 110, or a functional fragment and/or functional variant thereof.

In some embodiments, the aptameric nucleotide sequence is modified to be more resistant to denaturation according to methods known in the art. For example, the aptameric nucleotide sequence may be modified to include a locked nucleic acid (LNA), 2′-O-methyl nucleotides, or 2′-fluoro-deoxyribonucleotides. In certain embodiments, the aptameric nucleotide sequence is modified to comprise a locked nucleic acid (LNA), 2′-O-methyl nucleotides, or 2′-fluoro-deoxyribonucleotides.

### Linkers

According to embodiments, the spacing of the aptameric nucleotide sequences is optimized to allow symmetrical interaction between the trimeric aptamer and the homotrimeric protein.

In certain embodiments, the linker separates an aptameric nucleotide sequence from the respective terminal end of the connector at a distance of up to 25 nm. In other embodiments, the linker has a span of up to 25 nm, 20.5 nm, 18 nm, 15 nm, 12 nm, 10 nm, 8 nm, 5 nm, or 0 nm. In other embodiments, the linker has a span of 5 nm to 25 nm, 10 nm to 20.5 nm, or 10 nm to 18 nm. In some embodiments, the linker has the same span length for each aptameric nucleotide sequence on its respective terminal end of the connector arm. In other embodiments, the span length of each linker is different for each aptameric nucleotide sequence.

According to embodiments, the linker is a linear carbon molecule. In such embodiments, the linear carbon molecule comprises up to 15 linear alkane chains each possessing up to 12 carbon atoms. In other embodiments, the linker is a linear polyethylene glycol chain possessing up to 55 ethylene glycol units. In further embodiments, the linker is an unstructured flexible single stranded nucleic acid sequence of up to 30 deoxythymidine or other bases. In other embodiments, the linker is an unstructured flexible single stranded nucleic acid sequence of 10 to 30, 15 to 30, 20 to 30, or 25 to 30 deoxythymidine or other bases. In certain embodiments, the linker is a 15-thymine nucleic acid sequence.

According to some embodiments, the 3′ arm of the connector may comprise a linker. In such embodiments, the linker molecule has a span of up to 25 nm, 20.5 nm, 18 nm, 15 nm, 12 nm, 10 nm, 8 nm, 5 nm, or 0 nm. In other embodiments, the linker has a span of 5 nm to 25 nm, 10 nm to 20.5 nm, or 10 nm to 18 nm. In some embodiments, the linker has the same span length as each linker between an aptameric nucleotide sequence and its respective terminal end of the connector. According to embodiments, the linker is a linear carbon molecule. In such embodiments, the linear carbon molecule comprises up to 15 linear alkane chains each possessing up to 12 carbon atoms. In other embodiments, the linker is a linear polyethylene glycol chain possessing up to 55 ethylene glycol units. In further embodiments, the linker is an unstructured flexible single stranded nucleic acid sequence of up to 30 deoxythymidine or other bases. In other embodiments, the linker is an unstructured flexible single stranded nucleic acid sequence of 10 to 30, 15 to 30, 20 to 30, or 25 to 30 deoxythymidine or other bases.

Persons of skill in the art will readily appreciate the linkers that can be used. For example, in certain embodiments, the linker is Spacer Phosphoramidite 18 (https://www.glenresearch.com/spacer-modifiers/10-1918.html) or Spacer C12 CE Phosphoramidite (https://www.glenresearch.com/spacer-modifiers/10-1928.html).

### Connector

According to embodiments, the homotrimeric DNA aptamer comprises a connector having up to three variable branches/arms and a 3′branch/arm, wherein each arm comprises a terminal end that links to one of the aptameric nucleotide sequences to form a trimeric configuration. The connector is a molecule connecting the aptameric nucleotide sequences via optional linkers. According to embodiments, the connector molecule is a trebler, or another branching chemical group, with multiple arms/branches.

According to certain embodiments, the connector is a trebler phosphoramidite. In certain embodiments, the homotrimeric DNA aptamer is a symmetrical trimeric construct. In such embodiments, the connector is a Symmetric Trebler molecule (Glen Research 10-1922).

Further embodiments provide for orientation control. Given that aptamers and their targets are known to bind in a preferred relative orientation, the homotrimeric DNA aptamer constructs allow for the attachment of homomeric aptamers in either the 5′-3′ or 3′-5′ orientation relative to the central connector. This allows for the aptamers to be arranged in an optimal orientation for epitope binding. According to embodiments, the aptameric nucleotide sequence may be connected to the connector from either the 5′, 3′ or at an internal position of the nucleotide sequence. In other embodiments, the homotrimeric DNA aptamer constructs comprise aptameric nucleotide sequences that are in the same 5′ to 3′ orientation relative to the connector. In further embodiments, the homotrimeric DNA aptamer constructs comprise aptameric nucleotide sequences that are in the same 3′ to 5′ orientation relative to the connector.

### 3′ Branch/Arm

According to embodiments, the 3′ branch/arm of the homotrimeric DNA aptamer construct provides a multipurpose attachment site on the 3′ end of the trebler connector. In such embodiments, the 3′ arm may be functionalized with a variety of other molecules to enable i) surface immobilization or crosslinking via biotin, amine, thiol or digoxigenin molecules, ii) attachment of reporter elements such as fluorophores, enzymes (HRP), nucleic acid amplification primers (RCA, PCR, LAMP, etc.) or iii) drug delivery such as small molecule drugs, antimicrobial agents, and gene therapeutics.

In certain embodiments, the 3′ arm of the homotrimeric DNA aptamer is attached to a functional molecule selected from an aptamer, a reporter molecule, or a crosslinker. In certain embodiments, the functional molecule is an aptameric nucleotide sequence identical to one or more of the aptameric nucleotide sequences. In other embodiments, the functional molecule is a reporter molecule selected from an antigen, an enzyme, and a fluorescent molecule. In further embodiments, the functional molecule is a crosslinker selected from thiol, amide, biotin, digoxigenein, azide, alkyne, carboxyl, and a click-chemistry-based crosslinker.

### Binding Affinity & Specificity

In various embodiments, the homotrimeric DNA aptamer described herein, and/or compositions or formulations comprising these trimeric aptamers, exhibit enhanced affinity for a target molecule, a homotrimeric protein. According to certain embodiments, the homotrimeric DNA aptamers described herein exhibit a synergistic binding affinity to a target. In particular, it was unexpectedly found that the homotrimeric DNA aptamer, i.e., the exemplary TMSA52 aptamer, exhibited an enhanced or synergistic affinity for SARS-CoV-2 spike protein and its variants by at least two order of magnitude over its corresponding monomeric or dimeric form. The unexpectedly enhanced affinity of the homotrimeric DNA aptamer was observed to display cooperativity between each branch in binding the target molecule.

### Methods and Uses

### Methods of Treatment

The homotrimeric aptamer of the present disclosure is useful in a variety of applications including, but not limited to, methods for the treatment of a disorder or disease. In particular, the homotrimeric aptamer described herein provides a method for treating a SARS-CoV-2 infection in a subject, comprising administering to a subject the homotrimeric aptamer of the invention. In other embodiments, the homotrimeric aptamer described herein provides a method for neutralization of a SARS-CoV-2 virus.

Further provided are pharmaceutical compositions, comprising a homotrimeric aptamer of the invention and one or more pharmaceutically acceptable excipients. In certain embodiments, the present invention provides for pharmaceutical compositions comprising an effective amount of a homotrimeric aptamer and one or more pharmaceutically acceptable carriers, diluents and/or excipients. If desired, other active ingredients may be included in the compositions, for example, additional immune stimulating compounds, standard therapeutics, vaccines or the like.

The pharmaceutical compositions can be formulated for administration by a variety of routes. For example, the compositions can be formulated for oral, topical, rectal, nasal or parenteral administration or for administration by inhalation or spray. The term parenteral as used herein includes subcutaneous injections, intravenous, intramuscular, intrathecal, intrasternal injection or infusion techniques. Intranasal administration to the subject includes administering the composition to the mucous membranes of the nasal passage or nasal cavity of the subject.

In some embodiments, the pharmaceutical compositions are formulated for mucosal administration. Mucosal administration may include, for example, oral, intranasal, aerosol, rectal or vaginal administration. The preparations for mucosal administration include transdermal devices, aerosols, creams, lotions or powders pending on the mucosal site. In certain embodiments, the pharmaceutical compositions are formulated for intranasal or pulmonary administration. In some embodiments, the pharmaceutical compositions are formulated for rectal or vaginal administration.

Compositions formulated as aqueous suspensions may contain the homotrimeric aptamer in admixture with one or more suitable excipients, for example, with suspending agents, such as sodium carboxymethylcellulose, methyl cellulose, hydropropylmethylcellulose, sodium alginate, polyvinylpyrrolidone, hydroxypropyl-β-cyclodextrin, gum tragacanth and gum acacia; dispersing or wetting agents such as a naturally-occurring phosphatide, for example, lecithin, or condensation products of an alkylene oxide with fatty acids, for example, polyoxyethyene stearate, or condensation products of ethylene oxide with long chain aliphatic alcohols, for example, hepta-decaethyleneoxycetanol, or condensation products of ethylene oxide with partial esters derived from fatty acids and a hexitol for example, polyoxyethylene sorbitol monooleate, or condensation products of ethylene oxide with partial esters derived from fatty acids and hexitol anhydrides, for example, polyethylene sorbitan monooleate. The aqueous suspensions may also contain one or more preservatives, for example ethyl, or n-propyl p-hydroxy-benzoate, one or more colouring agents, one or more flavouring agents or one or more sweetening agents, such as sucrose or saccharin.

In certain embodiments, the pharmaceutical compositions may be formulated as oily suspensions by suspending the homotrimeric aptamer in a vegetable oil, for example, arachis oil, olive oil, sesame oil or coconut oil, or in a mineral oil such as liquid paraffin. The oily suspensions may contain a thickening agent, for example, beeswax, hard paraffin or cetyl alcohol. These compositions can be preserved by the addition of an anti-oxidant such as ascorbic acid.

In certain embodiments, the pharmaceutical compositions may be formulated as a dispersible powder or granules, which can subsequently be used to prepare an aqueous suspension by the addition of water. Such dispersible powders or granules provide the homotrimeric aptamer in admixture with one or more dispersing or wetting agents, suspending agents and/or preservatives. Suitable dispersing or wetting agents and suspending agents are exemplified by those already mentioned above. Additional excipients, for example, colouring agents, can also be included in these compositions.

Pharmaceutical compositions of the invention may also be formulated as oil-in-water emulsions in some embodiments. The oil phase can be a vegetable oil, for example, olive oil or arachis oil, or a mineral oil, for example, liquid paraffin, or it may be a mixture of these oils. Suitable emulsifying agents for inclusion in these compositions include naturally-occurring gums, for example, gum acacia or gum tragacanth; naturally-occurring phosphatides, for example, soy bean, lecithin; or esters or partial esters derived from fatty acids and hexitol, anhydrides, for example, sorbitan monoleate, and condensation products of the said partial esters with ethylene oxide, for example, polyoxyethylene sorbitan monoleate.

In certain embodiments, the pharmaceutical compositions may be formulated as a sterile injectable aqueous or oleaginous suspension according to methods known in the art and using suitable one or more dispersing or wetting agents and/or suspending agents, such as those mentioned above. The sterile injectable preparation can be a sterile injectable solution or suspension in a non-toxic parentally acceptable diluent or solvent, for example, as a solution in 1,3-butanediol. Acceptable vehicles and solvents that can be employed include, but are not limited to, water, Ringer's solution, lactated Ringer's solution and isotonic sodium chloride solution. Other examples include, sterile, fixed oils, which are conventionally employed as a solvent or suspending medium, and a variety of bland fixed oils including, for example, synthetic mono- or diglycerides. Fatty acids such as oleic acid can also be used in the preparation of injectables.

Optionally the pharmaceutical compositions may contain preservatives such as antimicrobial agents, anti-oxidants, chelating agents, and inert gases, and/or stabilizers such as a carbohydrate (e.g. sorbitol, mannitol, starch, sucrose, glucose, or dextran), a protein (e.g. albumin or casein), or a protein-containing agent (e.g. bovine serum or skimmed milk) together with a suitable buffer (e.g. phosphate buffer). The pH and exact concentration of the various components of the composition may be adjusted according to well-known parameters.

Sterile compositions can be prepared for example by incorporating the homotrimeric aptamer in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile compositions, some exemplary methods of preparation are vacuum-drying and freeze-drying techniques which yield a powder of the active ingredient plus any additional desired ingredient from a previously sterile-filtered solution thereof.

Contemplated for use in certain embodiments of the invention are various mechanical devices designed for pulmonary or intranasal delivery of therapeutic products, including but not limited to, nebulizers, metered dose inhalers, powder inhalers and nasal spray devices, all of which are familiar to those skilled in the art.

Metered dose inhalers typically use a propellant gas and require actuation during inspiration. Dry powder inhalers use breath-actuation of a mixed powder. Nebulizers produce aerosols from solutions, while metered dose inhalers, dry powder inhalers, and the like generate small particle aerosols.

Some specific examples of commercially available mechanical devices include the ULTRAVENT® nebulizer (Mallinckrodt, Inc., St. Louis, Mo.), the ACORN II® nebulizer (Marquest Medical Products, Englewood, Colo.), the MISTY-NEB® nebulizer (Allegiance, McGraw Park, Ill.), the AEROECLIPSE® nebulizer (Trudell Medical International, Canada), the Accuspray™ nasal spray device (Becton Dickinson), the Mucosal Atomization Device (MAD300) (Wolfe Tory Medical), the OptiNose device (OptiNose, Oslo, Norway), the Nektar DPI system (Nektar Therapeutics, Inc., San Carlos, Calif.), the AERx pulmonary drug delivery system (Aradigm Corporation, Hayward, Calif.), the Spiros® device (Dura Pharmaceuticals), and the Respimat® device (Boehringer Ingelheim).

All such devices require the use of formulations suitable for the dispensing of the homotrimeric aptamer. Typically, each formulation is specific to the type of device employed and may involve the use of an appropriate propellant material, in addition to the usual diluents, adjuvants and/or carriers useful in therapy as would be understood by a worker skilled in the art. Also, the use of liposomes, microcapsules or microspheres, inclusion complexes, or other types of carriers is contemplated.

Other pharmaceutical compositions and methods of preparing pharmaceutical compositions are known in the art and are described, for example, in “Remington: The Science and Practice of Pharmacy” (formerly “Remington Pharmaceutical Sciences”); Gennaro, A., Lippincott, Williams & Wilkins, Philadelphia, Pa. (2000).

### Methods of Detection and Diagnosis

The homotrimeric aptamer of the present disclosure is useful in a variety of applications including, but not limited to, methods for detecting the presence of SARS-CoV-2 infection in a subject.

In one aspect, the homotrimeric aptamer is useful for universally detecting the presence of current and ongoing SARS-CoV-2 spike protein variants of concern in a biological sample. In some embodiments, the homotrimeric aptamer binds the SARS-CoV-2 spike protein with picomolar affinity or femtomolar affinity.

The term “detecting” as used herein includes quantitative or qualitative detection. In some embodiments, the sample comprises, but is not limited to, tissue samples, saliva, sputum, urine, blood, serum, other bodily fluids and/or secretions.

In some embodiments, detection of the target molecule is indicated by a fluorescent, colorimetric, electrochemical, surface plasmon resonance, spectroscopic, or radioactive signal. In some embodiments, detection of the target molecule is indicated by a fluorescent signal. In some embodiments, an increase in the fluorescence signal indicates presence of the target in the sample.

### Kits

In certain aspects of the invention, kits are provided comprising a container housing a composition comprising the homotrimeric aptamer.

### Pharmaceutical Kits

Certain embodiments of the invention provide for pharmaceutical kits comprising homotrimeric aptamer for use as a therapeutic. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale. The kit may optionally contain instructions or directions outlining the method of use or administration regimen for the homotrimeric aptamer.

When one or more components of the kit are provided as solutions, for example an aqueous solution, or a sterile aqueous solution, the container means may itself be an inhalant, syringe, pipette, eye dropper, or other such like apparatus, from which the solution may be administered to a subject or applied to and mixed with the other components of the kit.

The components of the kit may also be provided in dried or lyophilised form and the kit can additionally contain a suitable solvent for reconstitution of the lyophilised components. Irrespective of the number or type of containers, the kits of the invention also may comprise an instrument for assisting with the administration of the composition to a patient. Such an instrument may be an inhalant, nasal spray device, nebulizer, syringe, pipette, forceps, measured spoon, eye dropper or similar medically approved delivery vehicle.

### SARS-CoV-2 Detection Kits

According to certain embodiments, the homotrimeric aptamer can be combined into a test kit system. For example, the homotrimeric aptamer can be combined into a biosensor system or kit for use to detect SARS-CoV-2 spike protein variants of concern in point-of-care testing for screening, diagnostics and/or health monitoring, and instructions for use.

In some embodiments, the sample is a biological sample, and the presence of the SARS-CoV-2 spike protein target in the sample is indicative of, or associated, with a Covid-19 infection. Accordingly, provided is a method of detecting SARS-CoV-2 infection in a subject comprising testing a sample from the subject for the presence of the SARS-CoV-2 spike protein or variants thereof using the homotrimeric aptamer combined into a biosensor, biosensor system and/or kit, wherein presence of the SARS-CoV-2 spike protein or variants indicates that the subject has an infection.

In accordance with another aspect, there is provided a kit for detection of a target in a sample comprising the homotrimeric DNA aptamer combined into a biosensor or biosensor system and instructions for use.

To gain a better understanding of the invention described herein, the following examples are set forth. It will be understood that these examples are intended to describe illustrative embodiments of the invention and are not intended to limit the scope of the invention in any way.

### EXAMPLES

### Example 1: Preparation of Homotrimeric Aptamer TMSA52

Design of a symmetric homotrimeric aptamer for the trimeric spike protein of SARS-CoV-2. The aptamer used for this work is MSA52, a monomeric DNA aptamer MSA52 (FIG. 1B) discovered by the inventors through selection with variant S proteins. Impressively, MSA52 was found to universally recognize variants that were not analyzed in the original selection experiment, demonstrating that the aptamer is insensitive to emerging S protein mutations. Hence, MSA52 is an ideal candidate for COVID-19 recognition.

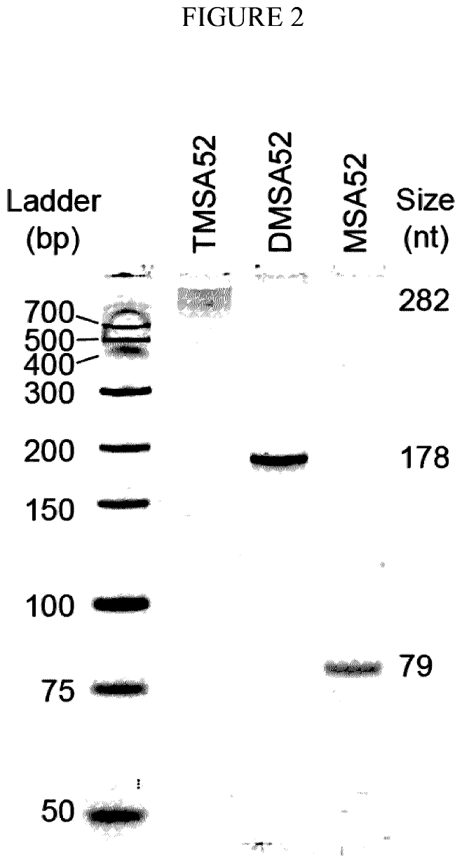

With the use of a 15-thymine linker and DNA synthesizer, the branched structural scaffold of a trebler was harnessed to synthesize a DNA molecule containing three identical MSA52 sequences (FIG. 1C), referred to herein as TMSA52. Analysis with 10% denaturing polyacrylamide gel electrophoresis (dPAGE) of chemically synthesized TMSA52 showed that TMSA52 was synthesized successfully in reference to monomeric and dimeric MSA52 sequences (FIG. 2).

### Materials and Reagents

DNA oligonucleotides listed in Table 4 were obtained from Yale University or Integrated DNA Technologies and purified using 10% denaturing polyacrylamide gel electrophoresis (dPAGE) containing 8 M urea. Sodium borohydride (NaBH⁴, 98%), sodium hexachloroiridate (III) hydrate (Na₃IrCl₆·xH₂O, M.W.=473.9), potassium phosphate monobasic (KH₂PO₄, ≥99%), sodium phosphate dibasic (Na₂HPO₄, ≥99%), potassium chloride (KCl, ≥99%), sodium chloride (NaCl, ≥99.5%), 4-(2-hydroxyethyl)-1-piperazineethanesulfonic acid (HEPES, ≥99%), magnesium chloride (MgCl₂, ≥99%), acetic acid (HOAc, ≥99.7%), 3,3′,5,5′-tetramethylbenzidine (TMB, >99%), sodium acetate (NaOAc, ≥99%), sulfuric acid (H₂SO₄, 95-98%), hydrogen peroxide solution (30% H₂O₂), dimethylformamide (DMF), streptavidin (Cat. No. SA101), bovine serum albumin (BSA, Cat. No. A7906), amylase (Cat. No. A1031), human IgG (Cat. No. 14506) and Tween-20 were all obtained from Sigma-Aldrich. The spike proteins of B.1.1.7 (Cat. No. SPN-C52H6), B.1.617.2 (Cat. No. SPN-C52He), B.1.617.1 (Cat. No. SPN-C52Hr), and B.1.1.529 (Cat. No. SPN-C52 Hz) SARS-CoV-2 variants expressed in human 293 cells (HEK293) were obtained from Acro Biosystems. The spike proteins of B.1.351 (Cat. No. 510333-1), B.1.429 (Cat. No. 101057) and P.1 (Cat. No. 100989-1) SARS-CoV-2 variants expressed in human 293 cells (HEK293) were obtained from BPS Biosciences Inc. The spike proteins for wild-type SARS-CoV-2 and SARS-CoV-1, the spike protein RBD of seasonal coronavirus 229E and OC43, the control lentiviruses, the pseudotyped lentiviruses expressing the spike proteins of wild-type, B.1.351, and P.1 SARS-CoV-2 were obtained from Dr. Matthew Miller's lab at McMaster University. The pseudotyped lentiviruses expressing the spike proteins of B.1.1.7 (Cat. No. 78112-1), B.1.617.1 (Cat. No. 78205-1), B.1.429 (Cat. No. 78172-1), B.1.617.2 (Cat. No. 78216-1), and B.1.1.529 (Cat. No. 78348-1) SARS-CoV-2 were purchased from BPS Bioscience. Nitrocellulose membranes (Cat. No. 10600125) were from GE Healthcare Inc. Nylon membranes (Cat. No. NEF994001PK) were obtained from PerkinElmer Inc. The pooled human saliva (Lot 31887) was from Innovative Research Inc (Novi, Michigan). T4 polynucleotide kinase (PNK) with 10× buffer was acquired from Thermo Scientific (Ottawa, Canada). [γ-³²P]-ATP was purchased from PerkinElmer. 96-well microtiter plates (clear, polystyrene, flat bottom) were from Celltreat Inc. Ultrapure water (Milli-Q System, Millipore) was used to prepare all aqueous solutions.

TABLE 4
Synthetic DNA oligonucleotides used in this research. All sequences are written
in a 5' to 3' direction. Italic T segments act as linkers.

| SEQ NO IDs | Name | Size (nt) | Sequence |
|---|---|---|---|
| 103 | MSA52 | 79 | TTACGTCAAG GTGTCACTCC GTAGGGTTTG GCTCCGGGCC |
| | | | TGGCGTCGGT CGTCTCTCGC GAAGCATCTC TTTGGCGTG |
| 108 | MSA52-B | 84 | Biotin-TTTTT TTACGTCAAG GTGTCACTCC GTAGGGTTTG |
| | | | GCTCCGGGCC TGGCGTCGGT CGTCTCTCGC GAAGCATCTC |
| | | | TTTGGCGTG |
| 111 | DMSA52 | 178 | TTACGTCAAG GTGTCACTCC GTAGGGTTTG GCTCCGGGCC |
| | | | TGGCGTCGGT CGTCTCTCGC GAAGCATCTC TTTGGCGTG |
| | | | TTTTTTTTTT TTTTTTTTTT TTACGTCAAG GTGTCACTCC |
| | | | GTAGGGTTTG GCTCCGGGCC TGGCGTCGGT CGTCTCTCGC |
| | | | GAAGCATCTC TTTGGCGTG |
| 112 | DMSA52-B | 183 | Biotin-TTTTT TTACGTCAAG GTGTCACTCC GTAGGGTTTG |
| | | | GCTCCGGGCC TGGCGTCGGT CGTCTCTCGC GAAGCATCTC |
| | | | TTTGGCGTG TTTTTTTTTT TTTTTTTTTT TTACGTCAAG |
| | | | GTGTCACTCC GTAGGGTTTG GCTCCGGGCC TGGCGTCGGT |
| | | | CGTCTCTCGC GAAGCATCTC TTTGGCGTG |
| 109 | TMSA52 | 282 | TTACGTCAAG GTGTCACTCC GTAGGGTTTG GCTCCGGGCC |
| | | | TGGCGTCGGT CGTCTCTCGC |
| | | | GAAGCATCTC TTTGGCGTG TTTTTTTTTT TTTTT-Trebler |
| 110 | TMSA52-B | 287 | TTACGTCAAG GTGTCACTCC GTAGGGTTTG GCTCCGGGCC |
| | | | TGGCGTCGGT CGTCTCTCGC |
| | | | GAAGCATCTC TTTGGCGTG TTTTTTTTTT TTTTT-Trebler- |
| | | | TTTTT-Biotin |
| 113 | AS | 40 | GCGAGAGACG ACCGACGCCA GGCCCGGAGC CAAACCCTAC |

### Example 2: Assessment of Binding Affinity

Dot-blot binding assays of the trimeric aptamer. The binding affinities of the trimeric aptamer for spike proteins and pseudoviruses were tested by dot-blot binding assays. Briefly, nitrocellulose and nylon membranes were first immersed in binding buffer (50 mM HEPES, 150 mM NaCl, 6 mM KCl, 2.5 mM MgCl₂, 2.5 mM CaCl₂), and 0.01% Tween-20, pH 7.4) for 1 h. Then, the radioactive TMSA52 (10 μL, <0.1 nM) in the binding buffer was denatured at 90° C. for 5 min and annealed at 22° C. for 10 min. Different concentrations of spike proteins or pseudoviruses (10 μL) were mixed with the TMSA52 and incubated at 22° C. for 30 min. The reaction mixture was then filtered consecutively through a nitrocellulose membrane, a nylon membrane, and a wetted Whatman paper filter assembled on a Whatman Minifold-1 96-well apparatus using a vacuum pump. The bound aptamer on the nitrocellulose membrane and the unbound aptamer on the nylon membrane were developed on a storage phosphor screen for 12 h and observed using a Typhoon 9200 imager (GE Healthcare). The dot intensity was quantified with Image J software to determine the bound fraction of aptamer, which was plotted against the concentration of spike proteins or pseudoviruses. The K_{d} values were obtained via non-linear curve fitting using Origin 2020 software by the equation Y=BₘₐₓX/(K_{d}+X), where Y refers to the bound fraction of aptamer, Bₘₐₓ represents the maximum bound fraction of aptamer, and X stands for the concentration of spike proteins or pseudoviruses. The mixture of ³²P-labelled TMSA52 (10 pM) and unlabeled AS (different concentrations) was prepared to test the blocking effect of AS on the binding affinity of TMSA52.

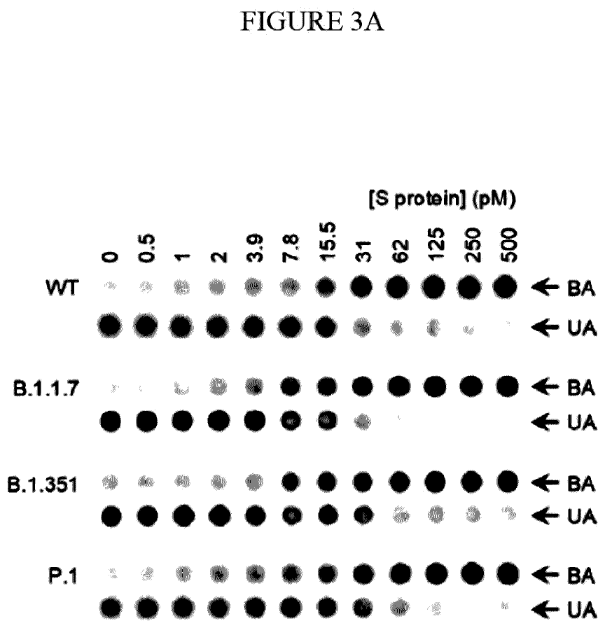

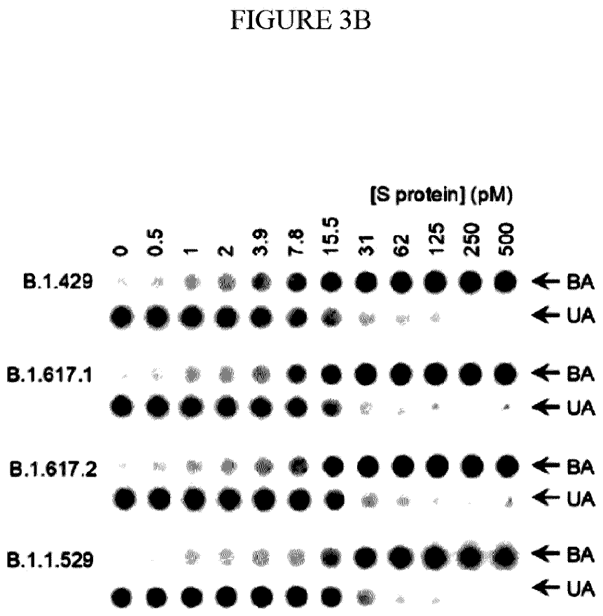

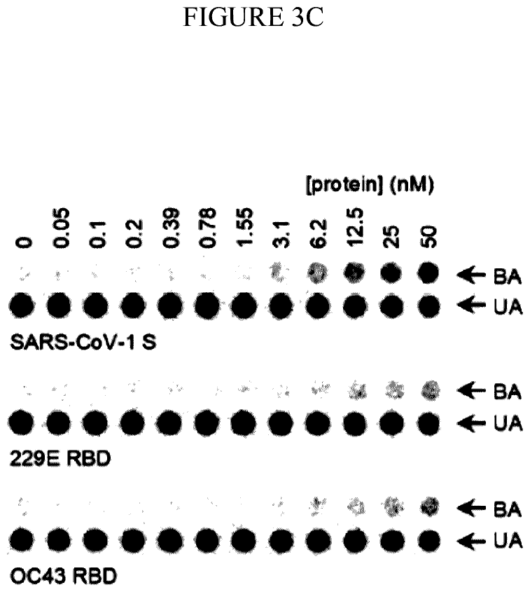

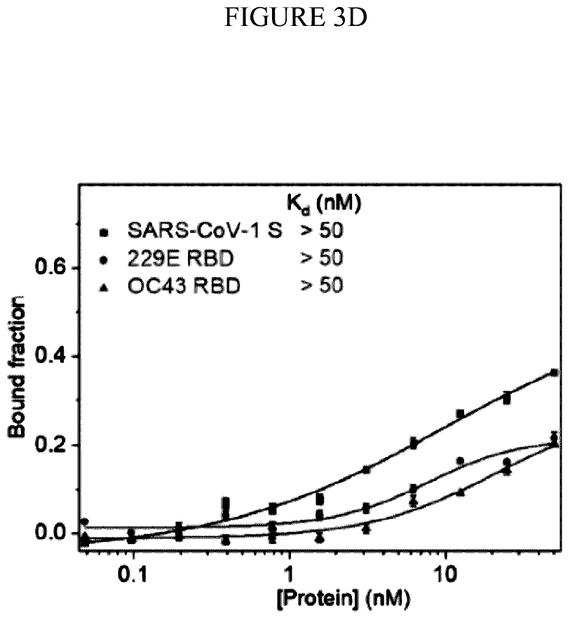

Assessment of binding affinity of TMSA52. Using a dot-blot assay, the binding affinity of TMSA52 (sequence listed in Table 4) was first tested for eight different SARS-CoV-2 spike protein variants, including the WT (Wild-Type), B.1.1.7 (Alpha), B.1.351 (Beta), P.1 (Gamma), B.1.429 (Epsilon), B.1.617.1 (Kappa), B.1.617.2 (Delta), and B.1.1.529 (Omicron) variants were tested using dot-blot assays (FIG. 3, panels A and B). After labeling with ³²P at the 5′ end, TMSA52 was incubated with different concentrations of spike protein variants to form an aptamer/protein complex. The aptamer/protein complex was retained by a nitrocellulose membrane, whilst the free aptamer was collected on a nylon membrane. The concentration of bound aptamer on the nitrocellulose membrane and unbound aptamer on the nylon membrane were determined by their radioactivity.

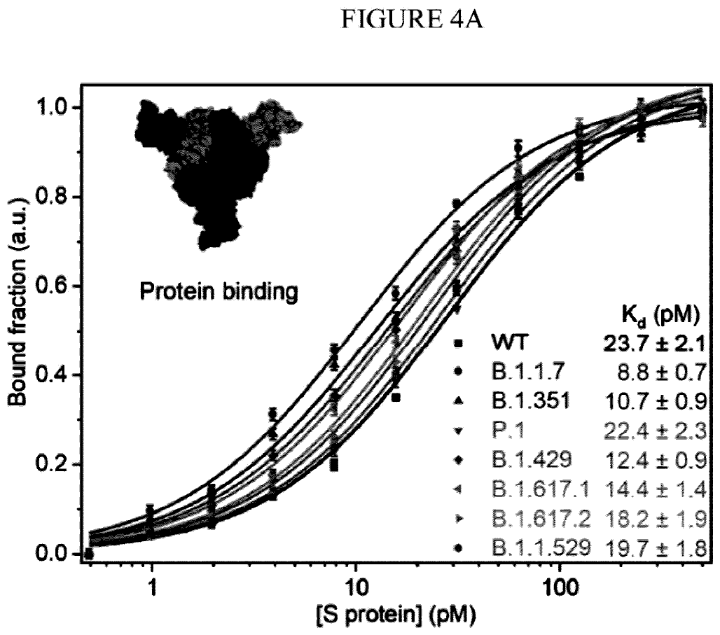

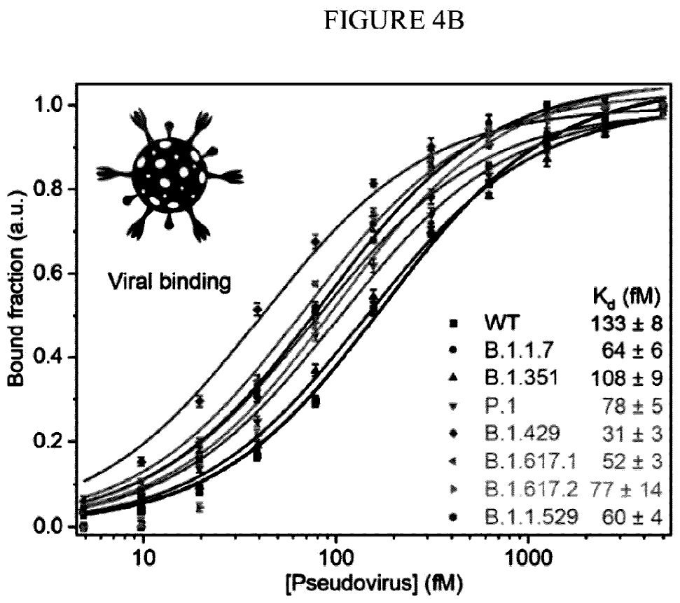

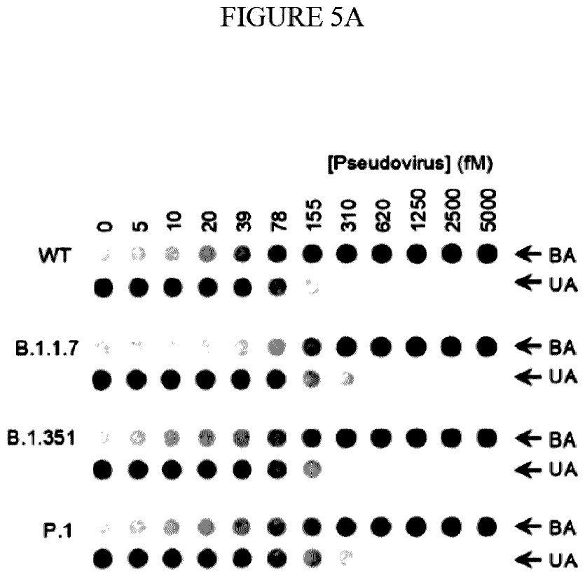

FIG. 4A plots the bound fraction of aptamer against the concentrations of each spike protein variant. The dissociation constants (K_{d} values) were obtained via non-linear curve fitting using the equation Y=BₘₐₓX/(K_{d}+X), where Y refers to the bound fraction of aptamer, Bₘₐₓ represents the maximum bound fraction of aptamer, and X stands for the concentration of spike proteins. The K_{d} values of TMSA52 for the eight spike protein variants ranged from 8.8 to 23.7 pM, which were approximately two orders of magnitude lower than for the corresponding monomeric aptamer MSA52. The significantly increased binding affinity was attributed to the superior trivalent interaction between the trimeric aptamer and the spike protein trimer.

TMSA52 was also tested for the binding of three control proteins including SARS-CoV-1 spike protein and spike RBD proteins of seasonal coronavirus 229E and OC43 (FIG. 3, panels C and D). The K_{d} values for the control proteins exceeded 50 nM, demonstrating the highly specific recognition ability of TMSA52 for the SARS-CoV-2 spike proteins.

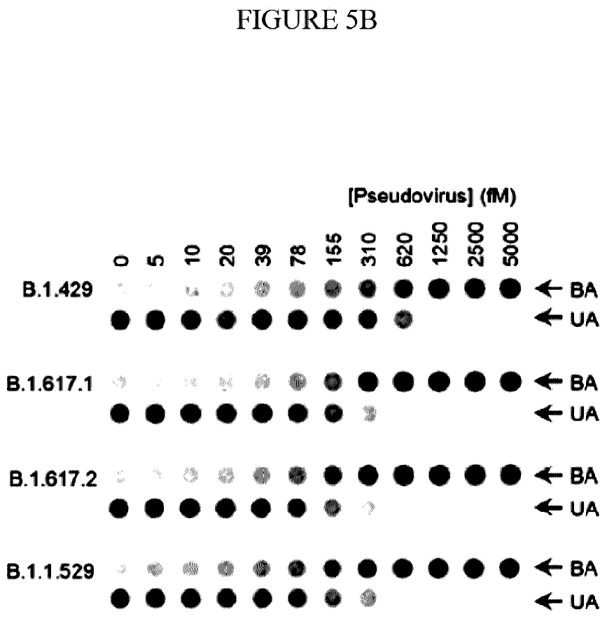

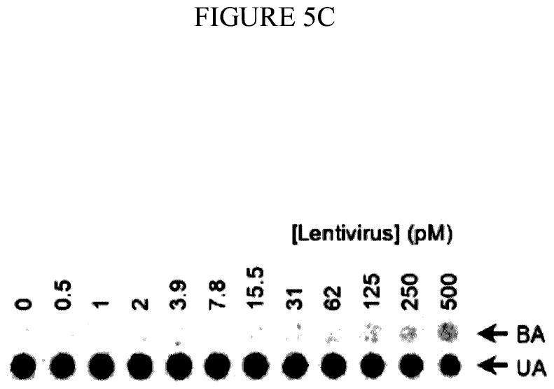

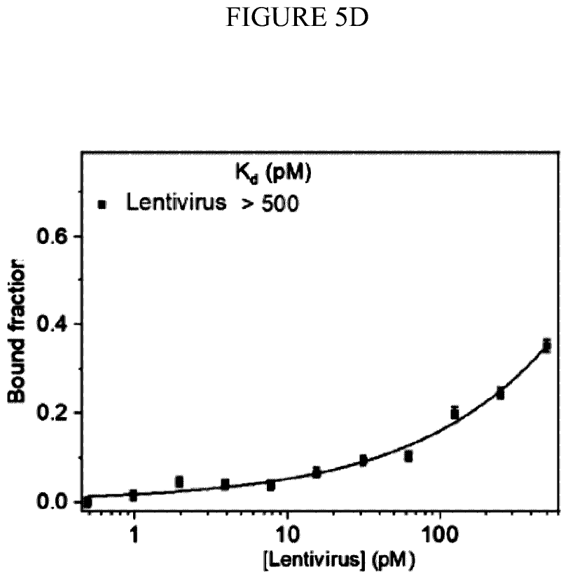

Following the same method, the binding affinities of TMSA52 were then tested for pseudotyped lentiviruses expressing the same spike protein variants, using a lentivirus without spike protein as a control (FIG. 5, panels A-C). The bound fraction of TMSA52 was plotted against the concentration of pseudoviruses to derive the K_{d} values (FIGS. 4B and 5D). The K_{d} values of TMSA52 for the eight pseudoviruses expressing different spike protein variants ranged from 31 to 133 fM, which was more than two orders of magnitude lower than for the monomeric aptamer MSA52. In contrast, the binding affinity for the control lentivirus was higher than 500 pM. Overall, the trimerization of MSA52 significantly increased the binding affinity for the recognition of all the SARS-CoV-2 spike protein variants.

### Example 3: Cooperative Binding by Trimeric Aptamers

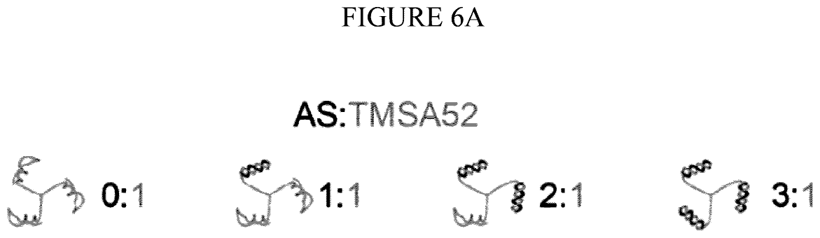

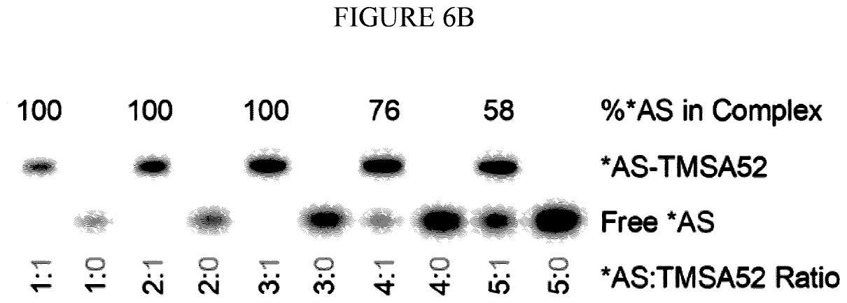

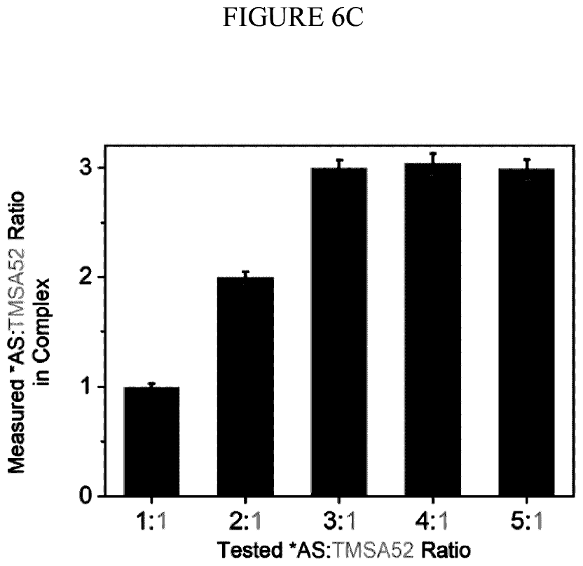

Cooperativity of binding by three aptamer arms. The next logical step was to validate the cooperative effect amongst the aptamer arms of TMSA52 for the binding of spike protein. By blocking the arms of TMSA52 with a 40-nt antisense (AS) DNA molecule (FIG. 6A), it can be assessed whether all three aptamer arms are required for the best possible binding. The precise AS: TMSA52 ratios were determined by an electrophoretic mobility shift assay, which used radioactive AS labeled with ³²P at the 5′ end. AS was mixed with TMSA52 in binding buffer at different ratios. After denaturation and annealing, the samples were analyzed by native polyacrylamide gel electrophoresis (nPAGE). As shown in FIG. 6 (panels B and C), ³²P-AS hybridized efficiently with TMSA52, but reached saturation at a ³²P-AS to TMSA52 ratio of 3:1. Further increases of ³²P-AS concentration resulted in no increase of binding number due to the limitation of three arms on each TMSA52.

### Materials and Reagents

Preparation of radioactive DNA. Trimeric aptamer TMSA52 was labeled with ³²P at the 5′ end using PNK according to a previously reported protocol with slight modifications.²⁴ Briefly, TMSA52 (2 μL, 1 μM) was mixed with [γ-³²P]-ATP (1 μL), 10×PNK reaction buffer A (1 μL), PNK (1 μL, 10 U/mL) and water (5 μL) in a 200-μL PCR tube followed by incubation at 37° C. for 20 min. The reaction mixture was then purified by 10% dPAGE containing 8 M urea. Radioactive AS was prepared as described for TMSA52.

Electrophoretic mobility shift assay. The hybridization of TMSA52 with AS was tested by an electrophoretic mobility shift assay. Briefly, different concentrations of ³²P-labelled AS (10 μL) were mixed with unlabeled TMSA52 (10 μL, 10 μM) in the binding buffer. The mixture was denatured at 90° C. for 5 min and annealing at 22° C. for 10 min. After the addition of the loading buffer, the samples were analyzed using 10% native PAGE. The image of the gel was developed on a storage phosphor screen for 12 h and observed using a Typhoon 9200 imager.

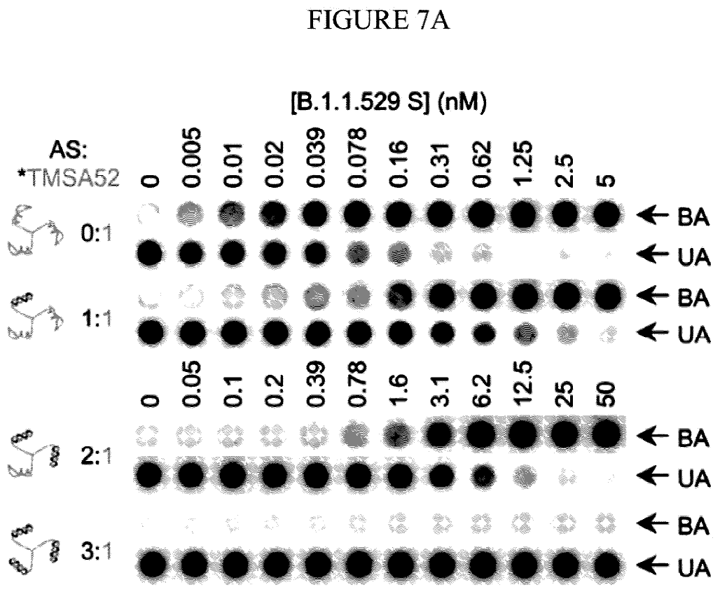

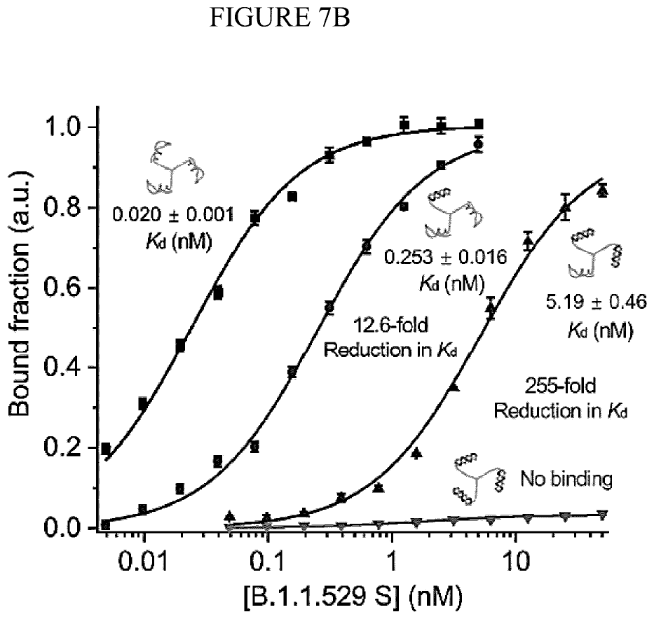

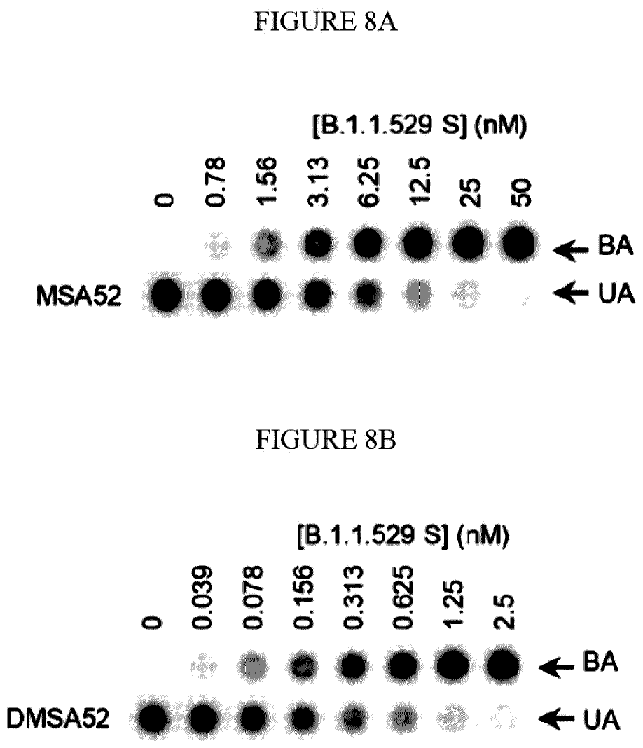

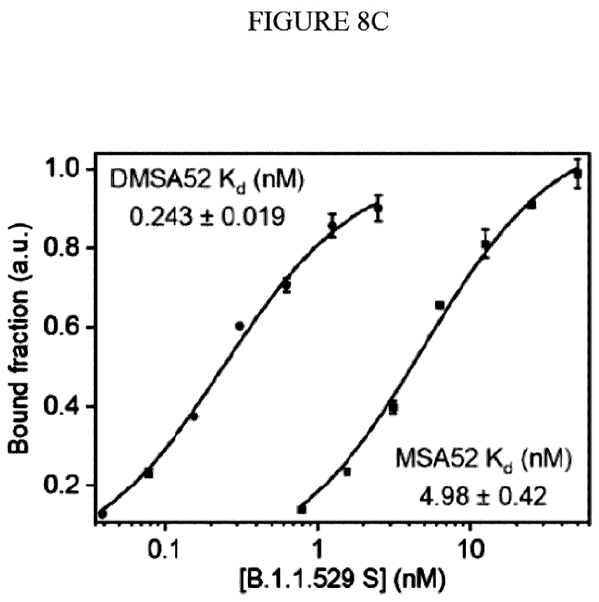

Assessment of binding affinity. Next the binding affinity of AS: TMSA52 complexes were investigated at different ratios for the spike protein. The B.1.1.529 variant spike protein was used for this experiment. The defined ratios of AS to ³²P-TMSA52 were mixed in binding buffer, followed by the addition of the B.1.1.529 spike protein. After a brief incubation at ambient temperature, the mixtures were analyzed by dot-blot assays. As shown in FIG. 7 panels A and B, the binding activity of TMSA52 was gradually reduced by AS with the K_{d} values increasing from 0.020 nM (AS:TMSA52=0:1) to 0.253 nM (AS:TMSA52=1:1) and 5.19 nM (AS: TMSA52=2:1). These values were consistent with the binding affinities of dimeric (DMSA52) and monomeric (MSA52) aptamers for the B.1.1.529 spike protein (FIG. 8). When the AS: TMSA52 ratio reached 3:1, the binding affinity of TMSA52 for the spike protein was almost completely abolished. These results adequately demonstrate that three TMSA52 arms associate with the three spike protein subunits in a concerted fashion. These results are consistent with the improved binding affinity of spherical aptamer, icosahedral DNA nanocage, and net-shaped DNA nanostructures for SARS-CoV-2 spike protein.

### Example 4: Enzyme-Linked Aptamer Binding Assay (ELABA)

Design of an enzyme-linked aptamer binding assay. The universal recognition for spike variants by TMSA52 offers a solution to a significant COVID-19 complication—the continuing emergence of variants of concern. These variants have significantly hindered the sensitivity of current antigen-based rapid tests and increase the need for continuous adjustments with novel MREs. TMSA52 offers a promising breakthrough in COVID-19 detection, as it has been shown to recognize a vast array of SARS-CoV-2 variants.

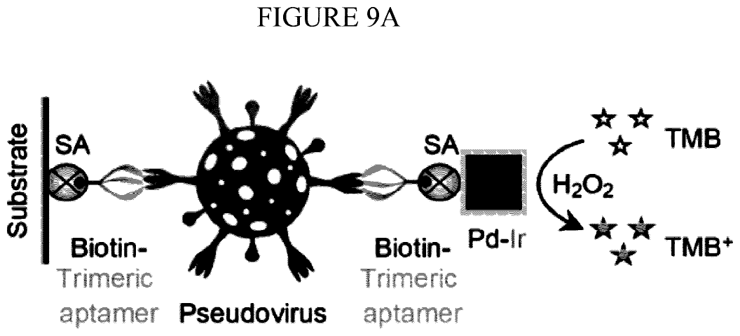

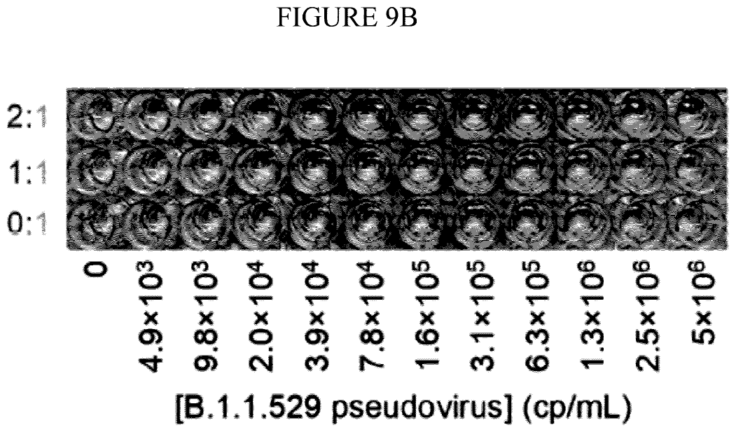

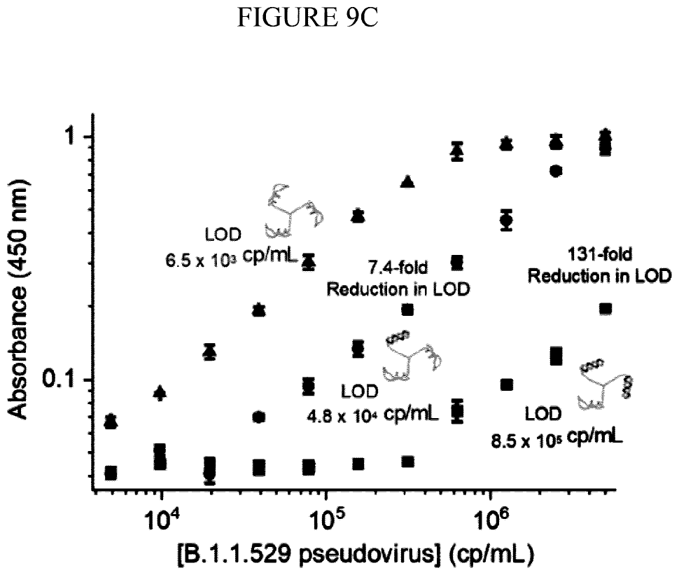

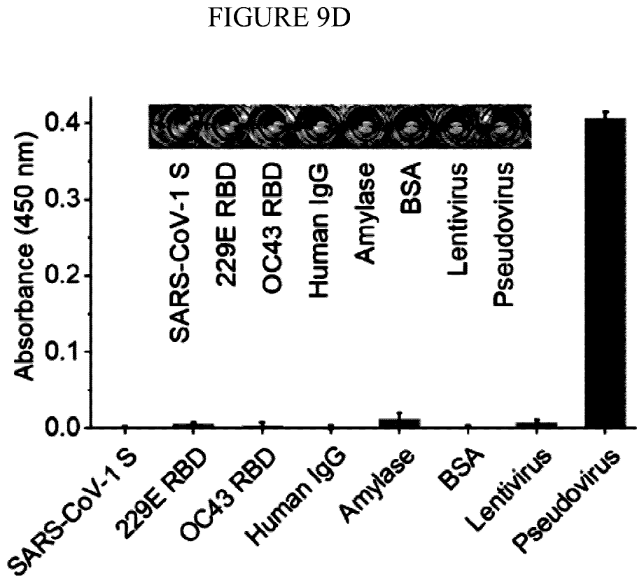

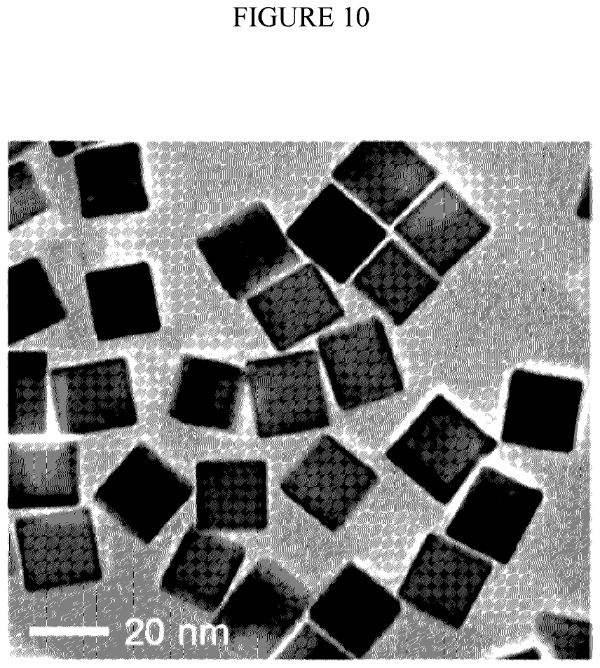

To employ the trimeric aptamer for the detection of all SARS-CoV-2 variants in an easy, lab-ready format, a sandwich assay was utilized employing nanozymes, entities known for their high peroxidase-mimicking activity (FIG. 9A). Nanozymes are popular candidates to provide colorimetric signal outputs and greatly improve detection sensitivity. Thus far, the nanozymes with the highest peroxidase-mimicking activity are Pd—Ir nanocubes, which display approximately three orders of magnitude higher catalytic activity than horseradish peroxidase (HRP). To conduct an enzyme-linked aptamer binding assay (ELABA), a biotinylated trimeric aptamer (TMSA52-B, Table 4) was first attached to a streptavidin-coated microtiter plate or Pd—Ir nanocubes (synthesized according to previously reported methods; see FIG. 10.) through the biotin/streptavidin interaction, which is one of the strongest biomolecular interactions. SARS-CoV-2 pseudovirus was then added to the aptamer-conjugated microtiter plate to bind with the immobilized trimeric aptamer. After washing, aptamer-conjugated Pd—Ir nanocubes were introduced to bind with the pseudovirus captured on the plate. The presence of SARS-CoV-2 pseudovirus variants leads to the immobilization of aptamer-conjugated Pd—Ir nanocubes through a sandwich structure, which efficiently catalyzes the oxidation of colorless TMB with H₂O₂ to generate blue oxidized products. H₂SO₄ is then used to convert the TMB from blue to yellow. The concentration of the pseudovirus, which is proportional to the absorbance at 450 nm, can be easily determined by a plate reader.

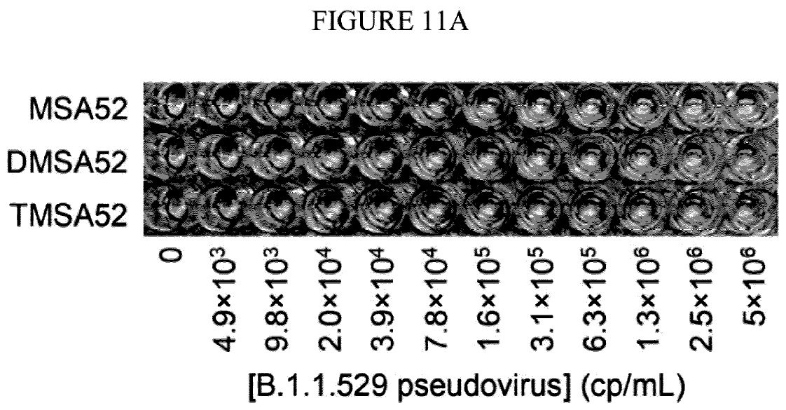

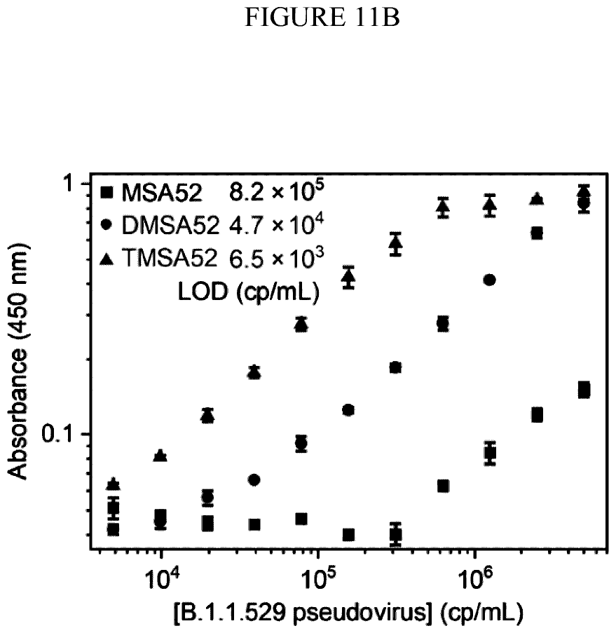

Using B.1.1.529 pseudovirus as a model target, the detection performance of trimeric (TMSA52-B), dimeric (DMSA52-B), and monomeric (MSA52-B) aptamer-based assays were compared. The ELABA procedures for dimeric and monomeric aptamers were the same as the above-described procedures for the trimeric aptamer, except for the substitution of TMSA52-B with DMSA52-B or MSA52-B. As shown in FIG. 11, the yellow (grey) intensity and absorbance at 450 nm increased proportionally with the concentration of the B.1.1.529 pseudovirus. The TMSA52-based method displayed a detection limit of 6.5×10³ cp/mL, which was 7.1-fold lower than DMSA52 (LOD 4.7×10⁴ cp/mL; LOD: limit of detection, 3-fold standard deviation of blank samples) and 126-fold lower than MSA52 (LOD 8.2×10⁵ cp/mL). The results demonstrate a significant improvement for the detection of pseudovirus by the trimerization of the original MSA52 aptamer.

To further verify the importance of trimeric binding for sensitive detection, AS was introduced to block the arms of TMSA52 prior to colorimetric detection. As shown in FIG. 9 panels B and C, the detection limit of the TMSA52-based assay increased from 6.5×10³ cp/mL to 4.8×10⁴ cp/mL and subsequently to 8.5×10⁵ cp/mL with the blocking of one and two TMSA52 arms, respectively. The results of increasing the AS: TMSA52 ratio were consistent with the DMA52 and MSA52-based assays (FIG. 11), demonstrating the importance of trimerization for enhancing the performance of biosensing assays.

Afterward, the specificity of the TMSA52-based assay for the detection of B.1.1.529 pseudoviruses was tested. SARS-CoV-1 spike, spike RBD of seasonal coronaviruses 229E and OC43, human IgG, amylase, BSA, and lentivirus were used as controls. The concentrations of control protein (10 nM) were approximately 5 orders of magnitude higher than B.1.1.529 pseudoviruses (120 fM). As shown in FIG. 9D, the assay was capable of specifically detecting B.1.1.529 pseudoviruses with a negligible signal response for the control proteins or lentivirus. These results strongly support the practicality of TMSA52 as a COVID-19 MRE as demonstrated by its exceptional binding affinity and specificity.

### Example 5: Detection of Pseudoviruses Spiked in Human Saliva

ELABA for the detection of psuedoviruses using trimeric aptamer and Pd—Ir nanocubes. Pseudoviruses were detected using the trimeric aptamer and Pd—Ir nanocube-based enzyme-linked aptamer binding assay (ELABA). Pd—Ir nanocubes with an edge length of 18 nm were prepared according to previously reported methods. The concentration of Pd—Ir nanocubes was 2.2 nM. Pd—Ir nanocubes were then conjugated with streptavidin through physical adsorption. Briefly, streptavidin (10 μL, 1 mg/mL) and mPEG-SH (10 μl, 100 μM) were added to the Pd—Ir nanocube solution (1 mL, 0.55 nM) and incubated at 22° C. for 1 h. After centrifuging at 10000 rpm for 5 min and washing once with PBST (PBS: 137 mM NaCl, 2.7 mM KCl, 8 mM Na₂HPO₄, and 2 mM KH₂PO₄, pH 7.4, containing 0.05% Tween-20), TMSA52-B (100 μL, 2 μM) in PBST was added and incubated at 22° C. for 30 min. Afterward, BSA (200 μL, 10% w/v) solution was added to the mixture and incubated at 22° C. for 1 h to block the Pd—Ir nanocubes. Aptamer-conjugated Pd—Ir nanocubes were pelleted by centrifuging at 10000 rpm for 5 min and washed twice with BSA solution (1 mL, 1% w/v). Thereafter, aptamer-conjugated Pd—Ir nanocubes were resuspended in PBST containing 10% w/v BSA, followed by storage at 4° C. for 12 h before use.

Subsequently, a 96-well microtiter plate was coated with streptavidin (100 μL, 5 μg/mL) in PBS by incubation at 4° C. for 12 h, followed by TMSA52-B conjugation (100 μL, 200 nM) in PBS at 22° C. for 30 min, and then blocking with BSA (100 μL, 2% w/v) in PBS at 37° C. for 1 h. After one wash with PBST (300 μL), different concentrations of pseudoviruses (100 μL) spiked in 25% saliva in PBST were added to the wells of aptamer-coated 96-well plate and incubated at 22° C. for 30 min. The plate wells were washed once with PBST (300 μL). Aptamer-conjugated Pd—Ir nanocubes (100 μL, 0.138 nM) in PBST were then added and incubated at 22° C. for 30 min. The wells were then washed four times with PBST (300 μL). Finally, TMB substrate solution (100 μL, 0.8 mM TMB, 2 M H₂O₂, 0.1 M HAcO/NaAcO buffer, pH 4) was added and reacted at 22° C. for 10 min, followed by terminating the catalytic reaction with H₂SO₄ (20 μL, 2 M). The absorbance of the oxidized TMB product at 450 nm was measured using a plate reader (Tecan, Switzerland). The protocols of MSA52 or DSA52-based detection were the same as above-described with the substitution of biotinylated trimeric aptamer (TMSA52-B) with monomeric (MSA52-B) or dimeric (DSA52-B) aptamers. To conduct the AS and TMSA52 mixture-based detection, different concentrations of AS (50 μL) were mixed with TMSA52-B (50 μL, 400 nM) in PBST buffer, followed by denaturing at 90° C. for 3 min and annealing at 22° C. for 10 min. Then, the mixture was used to substitute the biotinylated trimeric aptamer for the detection of pseudovirus as described above. To detect SARS-CoV-2 in clinical saliva samples by ELABA, saliva was first diluted by PBST to 25%, then added to the aptamer-coated plates and detected as described above.

ELABA for the detection of SARS-CoV-2 in patient saliva samples. Collection of saliva specimens was performed using a protocol approved by the Hamilton Integrated Research Ethics Board (HiREB Project #12636). Patients attending COVID-19 assessment centers at sites operated by Hamilton Health Sciences or St. Joseph's Healthcare in Hamilton, Ontario were invited to donate a supervised, self-collected, drool saliva sample immediately following collection of a nasopharyngeal swab (NPS). Saliva specimens were stored at 4° C. during transport (<72 hours) and subsequently long-term at −80° C. NPS specimens collected for standard COVID-19 screening were tested using a standard RT-PCR method by the Hamilton Regional Laboratory Medicine Program at St. Joseph's Healthcare Hamilton. NPS test results were used to identity candidate negative (60 patients, NS1-60), positive saliva specimens (27 patients, PS1-27) and to assign putative variants to each positive sample. The presence of SARS-CoV-2 in candidate saliva specimens was further confirmed using a saliva RT-PCR method described previously.

All patient saliva samples were diluted to 25% (v/v) using PBST and were tested using the ELABA method described above. Control reactions included a positive control (PC) containing 4×10⁴ cp/mL of B.1.1.529 pseudovirus spiked in commercial pooled human saliva and diluted to 25% (v/v) with PBST and a negative control (NC) consisting of commercial pooled human saliva diluted to 25% (v/v) with PBST. Data was plotted using a Receiver-Operator Characteristic plot and a cut-off was determined as the value that maximized the sum of Sensitivity and Specificity to determine the number of true positives and true negatives identified with the assay.

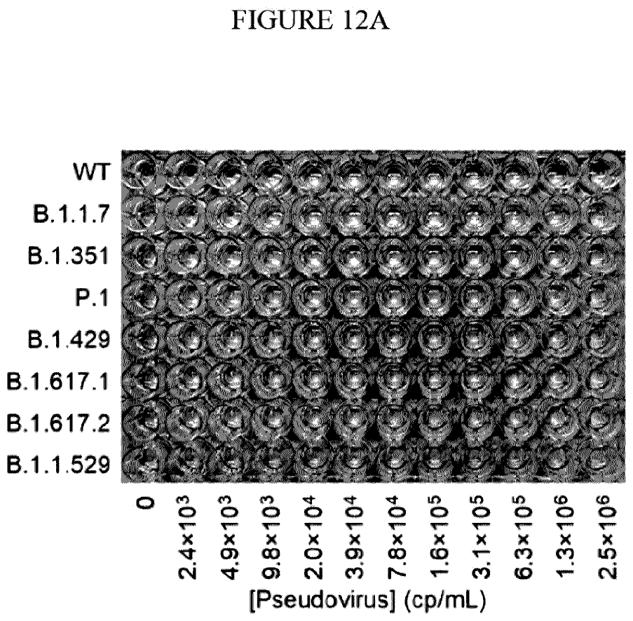

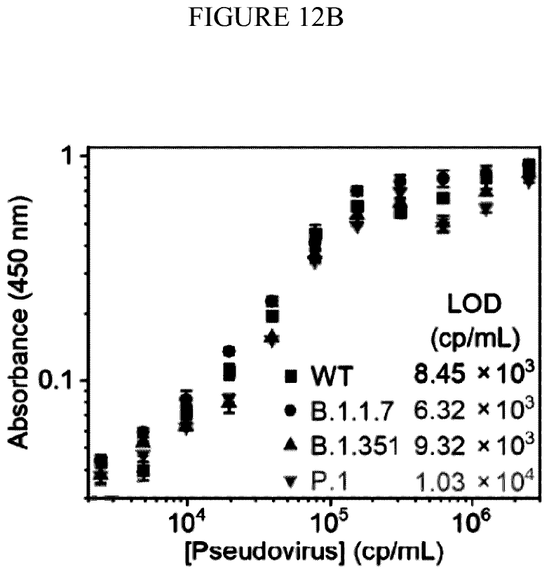

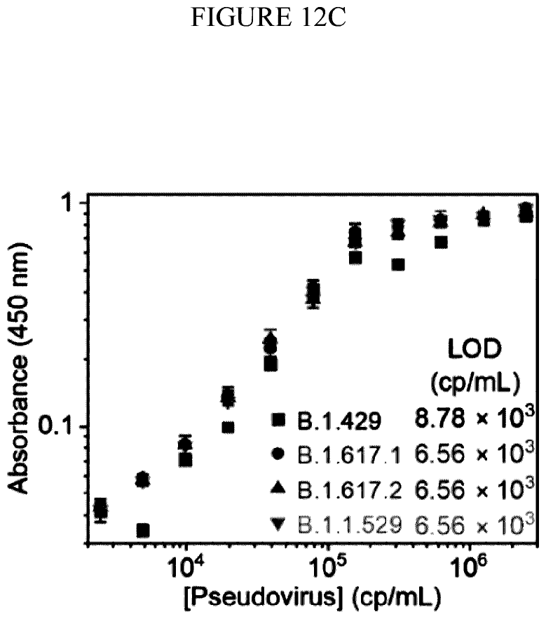

Detection of pseudoviruses spiked in human saliva. Next, the TMSA52-based biosensor was employed for the detection of eight SARS-CoV-2 spike variant pseudoviruses spiked in 25% pooled human saliva. Once again, the yellow (grey) intensity and absorbance at 450 nm increased proportionally with the pseudovirus concentration (FIG. 12). The limit of detection (LOD) ranged between 6.3×10³ to 1.0×10⁴ cp/mL for all eight pseudovirus variants, with the highest detection sensitivity for the B.1.1.7 variant and the lowest detection sensitivity for the P.1 variant. These results highlight the universal recognition capabilities of the TMSA52 aptamer.

### Example 6: Evaluation of the Biosensor Using Clinical Samples

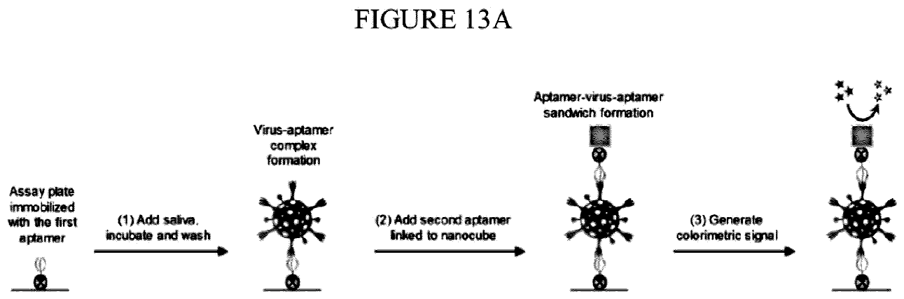

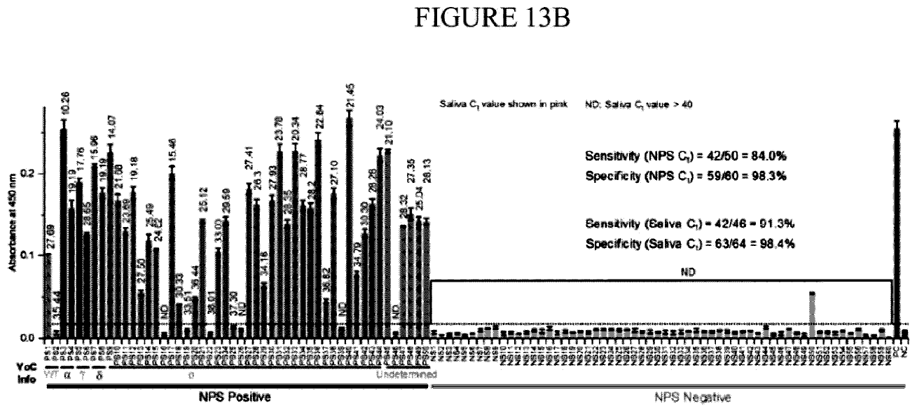

Evaluation of the biosensor using clinical samples. To evaluate the clinical utility of the sandwich assay employing the trimeric aptamer, a panel of 110 patient saliva samples were examined, including 50 NPS positive and 60 NPS negative samples. Table 5 provides details on each sample, including NPS and saliva-based Ct values obtained from RT-PCR along with the presumed variant (for positive samples). FIG. 13A shows the assay workflow. TMSA52-B was first immobilized to the surface of the microwell. Saliva samples were diluted to 25% using assay buffer and then incubated with the TMSA52-B aptamer (30 min) followed by a washing step to capture the virus on the microwell surface. The detection TMSA52-B aptamer, pre-bound to Pd—Ir nanoplates, was then added and allowed to incubate for 30 min prior to washing. TMB was then added and the signal was measured after 10 min. The total sample to readout time was 70 min. However, a total of 96 samples can be analyzed simultaneously by this method, producing an assay time of under one minute per sample.

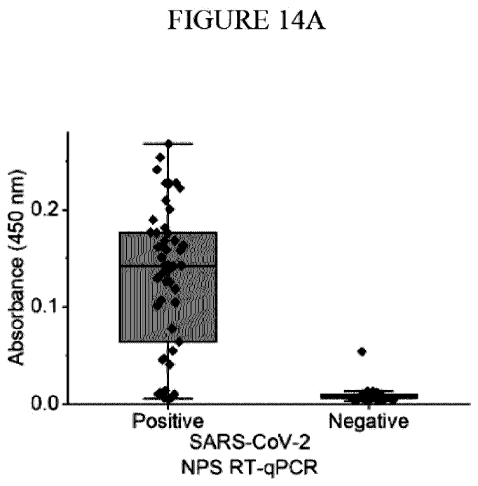

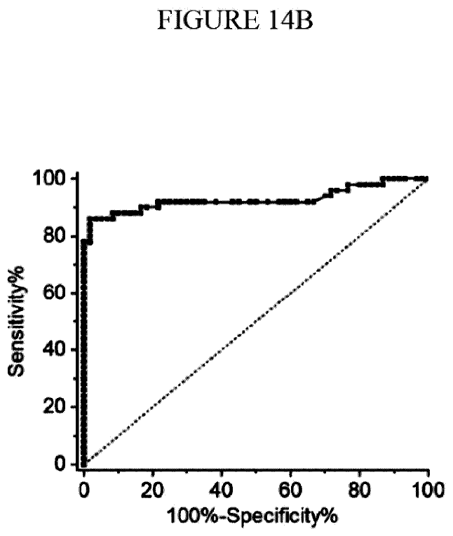

FIG. 13B shows the absorbance values measured for each of the individual positive (PS #) and negative (NS #) saliva samples, along with high (PC) and low (NC) controls. The Ct value for each positive saliva sample is shown above its respective bar. Based on a Receiver Operating Characteristic (ROC) curve (FIG. 14), the clinical cut-off absorbance (A450) value was determined to be 0.027, which resulted in a sensitivity of 84.0% true positives detected, a specificity of 98.3% true negatives detected, and an overall accuracy of 92.9%. It is noted that while each of the eight misidentified samples was identified as positive using RT-PCR of NPS samples (though all with Ct values over 33), four of these samples did not show detectable RNA in the corresponding saliva sample, hence the clinical sensitivity based on a comparison to saliva RT-PCR data rises to 91.3% if these four samples are considered as negatives.

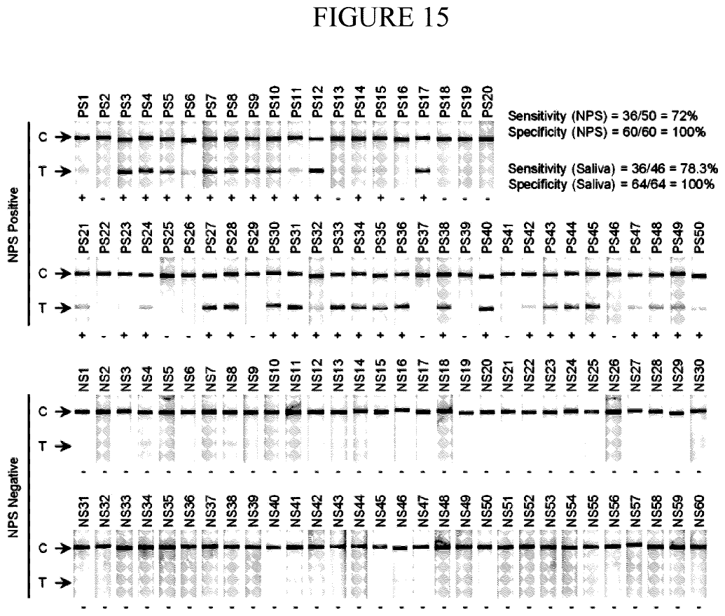

An important point from FIG. 13B is that the detection of SARS-CoV-2 in positive patient saliva does not depend on the variant. This clearly shows the key advantage of using the TMSA52 aptamer as it can produce positive signals regardless of the variant. The data also show that negative patient saliva samples do not contribute to significant background signals. This further supports the high selectivity of the TMSA52 aptamer, as it is insensitive to potential interferants that might be present in patient saliva. As a comparison, the 110 patient saliva samples were also tested by BTNX COVID-19 antigen rapid test. As shown in FIG. 15, the rapid test showed a detection sensitivity (NPS) of 72%, which was lower than the TMSA52-based ELABA method.

BTNX COVID-19 antigen rapid test for saliva samples. The saliva sample (30 μL) was mixed with detection buffer (270 μL), followed by addition to the well of BTNX COVID-19 antigen rapid test device (REF: CoV-19C25). After reaction for 20 min, the photograph of the test result was captured with a cellphone and processed with Photoshop 2020.

### Conclusion

Described herein is the most optimal aptamer thus far for SARS-CoV-2 recognition. To engineer the novel MRE, a trebler and linker system were adopted, whilst utilizing the pre-existing, universal aptamer MSA52. TMSA52 can recognize the most notable spike protein variants, including the wild-type, B.1.1.7 (Alpha), B.1.351 (Beta), P.1 (Gamma), B.1.429 (Epsilon), B.1.617.1 (Kappa), B.1.617.2 (Delta), and recent B.1.1.529 (Omicron) variants, with K_{d} values ranging from 8.8 to 23.7 pM. Compared to its monomeric and dimeric equivalents, TMSA52 exhibited increased binding affinity towards both the S protein and pseudovirus samples. The exceptional recognition was attributed to the symmetrical, multivalent interaction between the trimeric aptamer and spike protein trimer. For the application of the aptamer into a practical setting, a colorimetric assay for the detection of SARS-CoV-2 variants was developed using Pd—Ir nanocubes as peroxidase mimicking nanozymes for signal output. Eight pseudoviruses displaying different SARS-CoV-2 spike variants in pooled human saliva have been specifically identified with detection limits ranging from 6.3×10³ to 1.0×10⁴ cp/mL. Finally, the assay was applied to the detection of SARS-CoV-2 in patient saliva samples, providing a clinical sensitivity of 84.0% and specificity of 98.3% compared to RT-PCR of NPS samples.

The trimeric aptamer shape is a perfect fit for the trimeric S protein, granting it an affinity and specificity that is unique relative to any other aptamer published in the literature. TMSA52 holds great potential for broader diagnostic and therapeutic COVID-19 applications and, moving forward, should serve as the dominant choice of all SARS-CoV-2 MREs. The precision engineering of a trimeric aptamer specifically tailored to a trimeric protein provides a new approach for engineering high performing MREs for a wide range of target molecules.

TABLE 5
Comparative study of the positive COVID-19 saliva samples using
trimeric aptamer-based assay (ELABA), BTNX COVID-19 antigen
rapid test, NPS Ct (RT-PCR), and saliva Ct (RT-PCR).

| Sample ID | ELABA | Panbio | NPS Ct | Saliva Ct | Variant |
|---|---|---|---|---|---|
| PS1 | + | + | 19.23 | 27.69 | WT |
| PS2 | − | − | 33.16 | 35.44 | WT |
| PS3 | + | + | 22.26 | 10.26 | B.1.1.7 |
| PS4 | + | + | 21.32 | 19.19 | B.1.1.7 |
| PS5 | + | + | 13.37 | 17.76 | P.1 |
| PS6 | + | + | 13.48 | 28.65 | P.1 |
| PS7 | + | + | 14.19 | 15.96 | B.1.617.2 |
| PS8 | + | + | 19.44 | 19.19 | B.1.617.2 |
| PS9 | + | + | 16.87 | 14.07 | B.1.1.529 |
| PS10 | + | + | 1.18 | 21.68 | B.1.1.529 |
| PS11 | + | + | 14.27 | 23.69 | B.1.1.529 |
| PS12 | + | + | 21.40 | 19.18 | B.1.1.529 |
| PS13 | + | − | 17.33 | 27.50 | B.1.1.529 |
| PS14 | + | + | 17.38 | 25.49 | B.1.1.529 |
| PS15 | + | + | 27.40 | 24.62 | B.1.1.529 |
| PS16 | − | − | 29.60 | ND | B.1.1.529 |
| PS17 | + | + | 16.78 | 15.46 | B.1.1.529 |
| PS18 | + | − | 15.13 | 30.33 | B.1.1.529 |
| PS19 | − | − | 27.28 | 33.51 | B.1.1.529 |
| PS20 | + | − | 18.75 | 36.44 | B.1.1.529 |
| PS21 | + | + | 13.11 | 25.12 | B.1.1.529 |
| PS22 | − | − | 14.13 | 38.01 | BA.2 |
| PS23 | + | + | 32.48 | 33.00 | BA.2 |
| PS24 | + | + | 16.56 | 29.59 | BA.4 |
| PS25 | − | − | 16.30 | 37.30 | BA.4 |
| PS26 | − | − | 23.08 | ND | BA.4 |
| PS27 | + | + | 20.97 | 27.41 | BA.5 |
| PS28 | + | + | 18.22 | 26.30 | BA.5 |
| PS29 | + | − | 15.25 | 34.16 | BA.5 |
| PS30 | + | + | 15.68 | 27.93 | BA.5 |
| PS31 | + | + | 17.06 | 23.78 | BA.5 |
| PS32 | + | + | 22.26 | 28.35 | BA.5 |
| PS33 | + | + | 15.76 | 20.34 | BA.5 |
| PS34 | + | + | 15.08 | 28.77 | BA.5 |
| PS35 | + | + | 20.42 | 28.20 | BA.5 |
| PS36 | + | + | 18.59 | 22.84 | BA.5 |
| PS37 | + | − | 14.67 | 36.82 | BA.2.12.1 |
| PS38 | + | + | 19.49 | 27.10 | BA.2.12.1 |
| PS39 | − | − | 18.10 | ND | BA.2.12.1 |
| PS40 | + | + | 23.56 | 21.45 | BA.2.12.1 |
| PS41 | + | − | 12.38 | 34.79 | BA.2.12.1 |
| PS42 | + | + | 15.71 | 30.30 | BA.2.12.1 |
| PS43 | + | + | 18.21 | 28.28 | BA.2.12.1 |
| PS44 | + | + | 24.53 | 24.03 | BA.2.12.1 |
| PS45 | + | + | 26.14 | 21.10 | Undetermined |
| PS46 | − | − | 31.74 | ND | Undetermined |
| PS47 | + | + | 24.95 | 28.32 | Undetermined |
| PS48 | + | + | 16.50 | 27.35 | Undetermined |
| PS49 | + | + | 15.39 | 25.04 | Undetermined |
| PS50 | + | + | 14.40 | 28.13 | Undetermined |

Note:
‘+’ standards for positive results, whereas
‘−’ refers to negative results by trimeric aptamer-based assay.
PS: Positive saliva samples.

### Example 7: In Vitro Assessment of Aptamers in Neutralizing SARS-CoV-2

### In Vitro Neutralization Assay

Vero E6 cells (ATCC CRL-1586) were seeded at a density of 1.5×10⁴ cells/well in white flat-bottom TC-treated 96-well plates (Corning, 3917) and incubated at 37° C., 5% CO₂. On the day of seeding, at the end of the day, the medium was replenished with fresh DMEM fortified with 2% FBS, 1% Penicillin-Streptomycin, 1% HEPES (pH=7.3), and 1% Glutamax. The cells were then further incubated for 24 hours. Meanwhile, aptamers (TMSA52, Ir-TMSA52) and nanoplate (Ir only) were serially diluted down each column in an empty 96-well plate, starting with a 1:4 (BA.1) and 1:2 (MA10) dilution from a 1 μM stock solution and monoclonal antibody (S309) starting with 1 μg/ml. These aptamer dilutions were incubated with the ancestral strain and Omicron Variant (Lineage BA.1) of SARS-CoV-2 (330 plaque-forming units (PFU)/well) for 1 hour at 37° C., 5% CO₂. Following this incubation, the mixture was transferred onto the Vero E6 cells and re-incubated for 1 hour under the same conditions. The mixture was then replaced with identical dilutions of the aptamers and incubated for a further 72 hours at 37° C., 5% CO₂. Post-incubation, cell viability was ascertained using the CellTiter-Glo 2.0 Luminescent Cell Viability Assay Kit (Promega), where luminescence intensity was directly proportional to the number of viable cells. Luminescence was quantified using a BioTek Synergy H1 microplate reader, and neutralization titer was determined as the highest aptamer/antibody dilution that achieved a 50% reduction in luminescence compared to the virus control wells.

### Results:

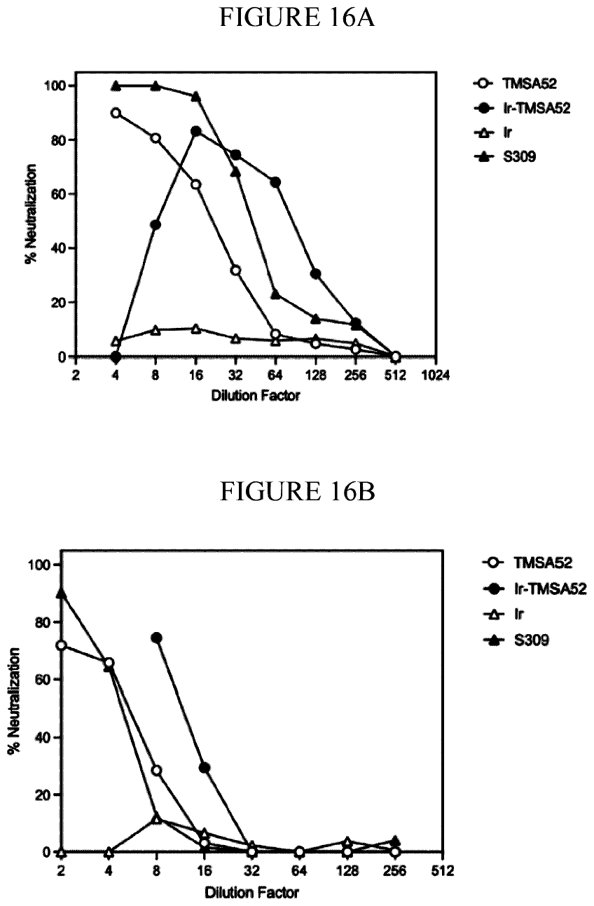

By utilizing a well-established microneutralization (MNT) assay, we assessed our aptamers in neutralizing SARS-CoV-2. Given the universality of our trimeric aptamer (TMSA52) or iridium nanoplate-scaffolded aptamer (Ir-TMSA52) in binding to the spike protein, we first quantified aptamer neutralization utilizing the immune evasive Omicron (BA.1) variant of SARS-CoV-2 and compared it to a clinically approved SARS-CoV-2 monoclonal antibody, S309 (FIG. 16A). In accordance with published data, S309 was capable of robustly neutralizing SARS-CoV-2 Omicron BA.1 (FIG. 16A, closed triangles). Impressively, both TMSA52 (FIG. 16A, open circles) and the iridium nanoplate-scaffolded Ir-TMSA52 (FIG. 16A, closed circles) both showed comparable neutralization activity to S309. The iridium nanoplate alone failed to confer any neutralizing activity (FIG. 16, open triangles). Given the robust neutralizing ability of our aptamers, we next assessed neutralization against a mouse-adapted variant of SARS-CoV-2 (MA10), preceding our downstream in vivo studies with the same virus strain. In accordance with the neutralization observed with SARS-CoV-2 Omicron BA.1, S309 was capable of robustly neutralizing SARS-CoV-2 MA10 (FIG. 16B, closed triangles). Impressively, both TMSA52 (FIG. 16B, open circles) and the iridium nanoplate-scaffolded Ir-TMSA52 (FIG. 16B, closed circles) both showed comparable neutralizing activity as S309. The iridium nanoplate alone failed to confer any neutralizing activity (FIG. 16B, open triangles).

### Conclusions:

Our aptamers, both the trimeric (TMSA52) and iridium nanoplate-scaffolded (Ir-TMSA52) demonstrate significant neutralization capabilities against SARS-CoV-2, matching the performance of the clinically-approved monoclonal antibody S309. These results highlight the robust potential of our aptamers as effective countermeasures against SARS-CoV-2 variants, showcasing their comparability to established therapeutic standards.

### Example 8: In Vivo Assessment of Aptamers in Protection Against Non-Lethal Challenge with Sars-COV-2

### Treatment Types and Delivery

Aptamers, nanoplate, and antibodies were synthesized in vitro and diluted to desired concentrations in nuclease-free water. A protective dose of intranasally (i.n.) administered S309 monoclonal antibody was previously determined (data not shown), and equimolar concentrations of aptamer were calculated for synthesis. Mice were deeply anesthetized by isoflurane inhalation and administered the desired treatment in a final volume of 40 μL (20 μL bolus to each nostril) two hours prior to infection.

### Virus Strains and Delivery

Wild-type SARS-CoV-2 (USA-WA1/2020) was serial passaged 10 times in BALB/c mice to generate SARS-CoV-2 (MA10), which was generously provided by Dr. Ralph Baric. Age-matched 6-8-week-old wild-type female BALB/c mice were purchased from Charles River Laboratories (Saint Constant, QC, Canada). Mice were deeply anesthetized by isoflurane inhalation, and infected i.n. with SARS-CoV-2 in a final volume of 40 μL (20 μL bolus to each nostril). Animals were housed in either a specific pathogen-free level B or a Containment Level 3 Facility at McMaster University, Hamilton, ON, Canada. All experiments were performed in accordance with institutional guidelines from the Animal Research and Ethics Board.

### Weight Loss and Clinical Scoring

Prior to treatment, mice were weighed to determine their initial weight as a benchmark. Mice were monitored for clinical signs and weight loss daily, with 80% of initial weight considered humane endpoint in accordance with institutional guidelines. Mice were scored for the presence of clinical signs, with one point being administered for ruffled fur, rapid breathing, hunched back, lethargy.

### SARS-CoV-2 Viral Burden Determination in Tissues

Vero E6 (CRL-1586, American Type Culture Collection (ATCC), Manassas, VA, United States) were cultured at 37° C. in Dulbecco's Modified Eagle medium (DMEM) supplemented with 10% fetal bovine serum (FBS), 1% HEPES (pH=7.3), 1 mM sodium pyruvate, 1% L-Glutamine and 100 U/mL of penicillin-streptomycin. Lungs were homogenized using a Bead Mill 24 homogenizer (ThermoFisher Scientific Waltham, MA, United States) and frozen at −80° C. Homogenates were thawed and clarified by centrifugation at 300×g. Homogenates were serially diluted 1:10 in low-serum DMEM supplemented with 2% FBS, 1% HEPES (pH=7.3), 1 mM sodium pyruvate, 1% L-Glutamine and 100 U/mL of penicillin-streptomycin. 100 μL of viral inoculum was transferred onto Vero E6 cells seeded the day before in 96-well plates (2.5×10⁴ cells per well). Wells were visually assessed for cytopathic effect at 72 hours post-infection using an EVOS M5000 microscope (ThermoFisher Scientific Waltham, MA, United States).

### Results:

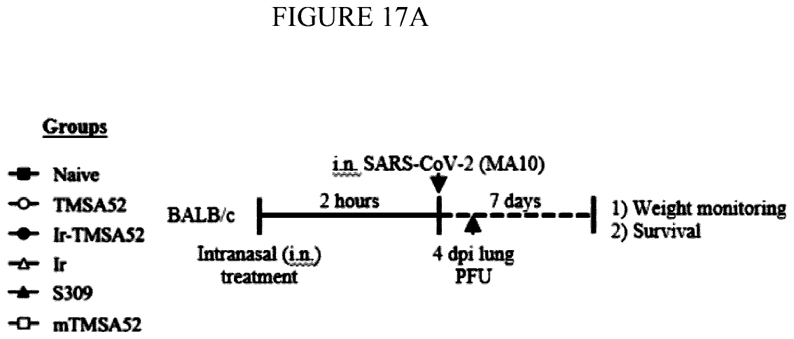

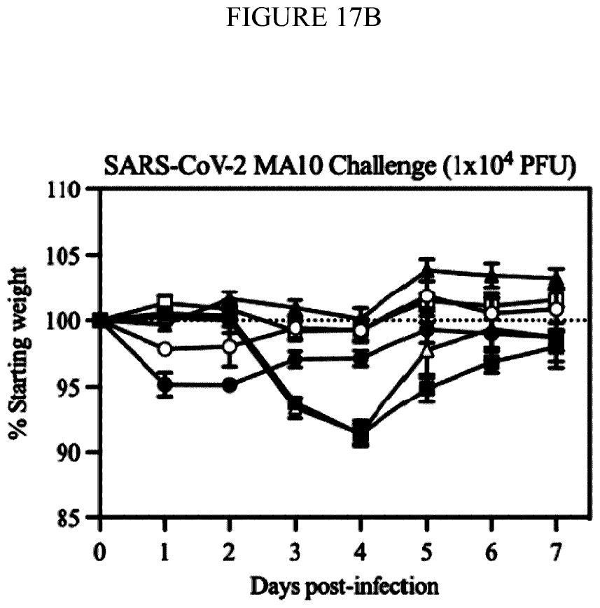

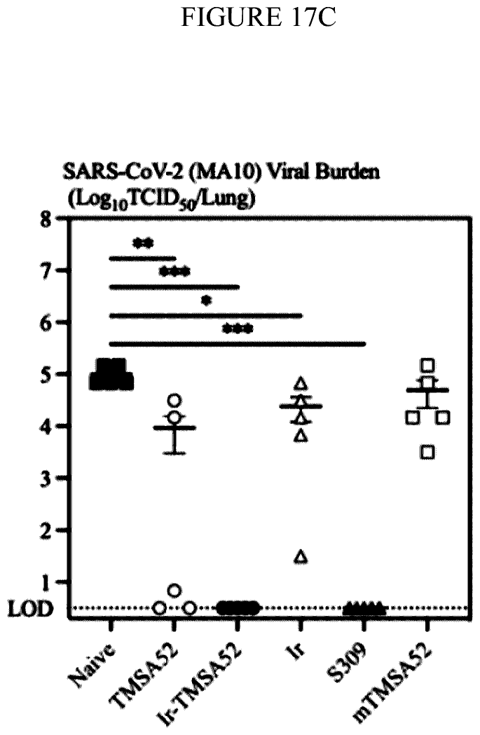

Effective protection against respiratory pathogens such as SARS-CoV-2 necessitates the delivery of prophylactic and therapeutic agents directly to the site of infection—the respiratory mucosa. To assess the prophylactic efficacy of our aptamer technology, female BALB/c mice (n=5-10 per group) were treated i.n. with a single bolus containing either trimeric aptamer (TMSA52, open circles), or the iridium nanoplate-scaffolded aptamer (Ir-TMSA52, closed circles) (FIG. 17A). As controls, a subset of animals was either treated with nuclease-free water (closed squares) or received a mutant version of TMSA52 (mTMSA52, open squares) that demonstrates reduced binding affinity to the SARS-CoV-2 receptor binding domain (RBD), or the iridium nanoplate alone (Ir, open triangles). As a positive control, a subset of animals was treated with a clinically approved SARS-CoV-2 monoclonal antibody (S309, closed triangles). Animals were subsequently i.n. infected with a sublethal dose of a mouse-adapted SARS-CoV-2 (1×10⁴ PFU) two hours after treatment. Animals were monitored daily for weight loss for 7 days, with a cohort sacrificed at 4 days post-infection (DPI) to enumerate lung viral burden.

Animals treated with nuclease-free water or those with the iridium nanoplate alone exhibited rapid and continued weight loss up to 4 days post-infection (FIG. 17B), at which animals began to recover. In stark contrast, TMSA52 and Ir-TMSA52 exhibited transient weight loss 2 days post-infection (≈5%), but quickly rebounded to pre-infection weights. No weight loss was observed in animals that received mutant aptamer or the control S309 monoclonal antibody.

To further evaluate the protection provided by i.n. aptamer delivery, viral burden was quantified 4 DPI by a 50% tissue-culture infectious dose (TCID₅₀) assay. As anticipated, animals treated with either the mutant aptamer or the iridium nanoplate alone had similarly high lung viral titers, comparable to nuclease-free water treated control animals (FIG. 17C). In stark contrast, 3/5 animals treated with TMSA52 had a >4 log₁₀ reduction in lung viral burden. Of note, animals that received Ir-TMSA52 had no quantifiable lung viral titers, comparable to animals treated with S309.

### Conclusions:

Intranasal delivery of both the trimeric aptamer (TMSA52) and the iridium nanoplate-scaffolded aptamer (Ir-TMSA52) offers robust protection against SARS-CoV-2. These treatments effectively mitigate morbidity and substantially reduce lung viral burden. The performance of both TMSA52 and Ir-TMSA52 was comparable to that of the clinically approved SARS-CoV-2 monoclonal antibody S309. These findings underscore the potential of our aptamer technology as an effective prophylactic strategy against SARS-CoV-2, exhibiting promising congruency to existing clinical standards.

### Example 9: In Vivo Assessment of Aptamers in Protection Against Lethal Challenge with Sars-COV-2

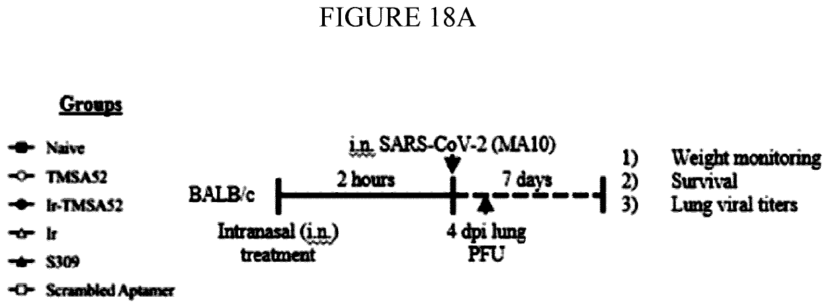

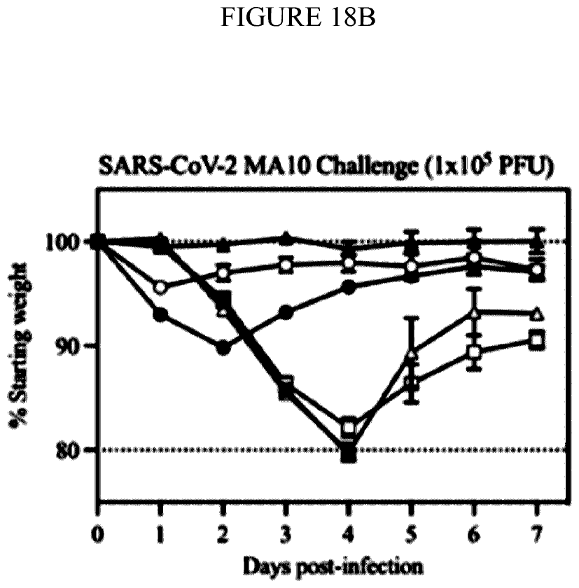

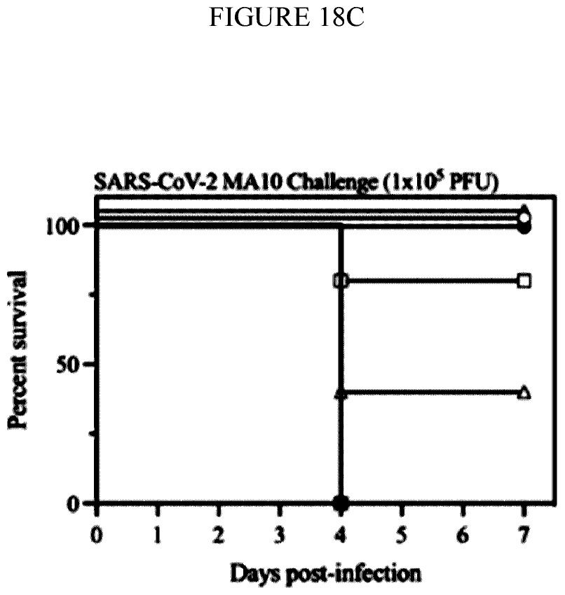

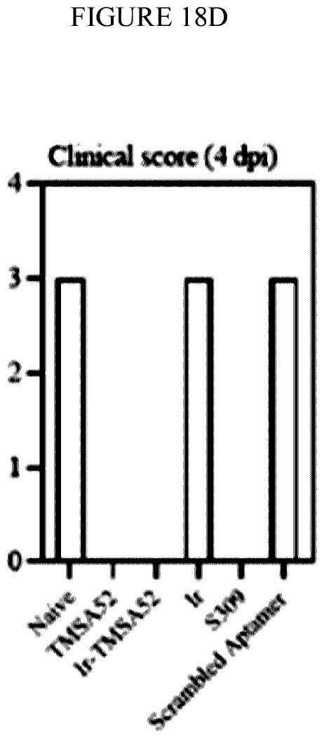

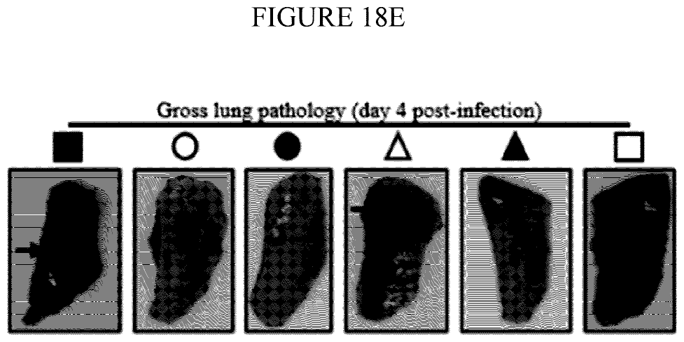

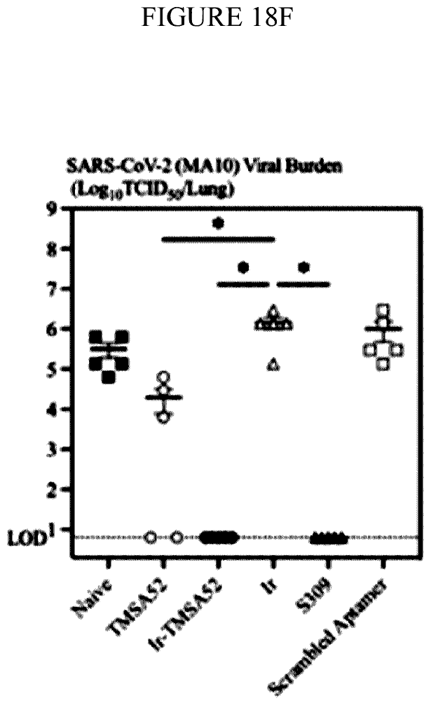

To further assess the prophylactic efficacy of our aptamer technology, female BALB/c mice (n=5-10 per group) were treated i.n. with a single bolus containing either trimeric aptamer (TMSA52, open circles), or the iridium nanoplate-scaffolded aptamer (Ir-TMSA52, closed circles) (FIG. 18A). As controls, a subset of animals was treated with either nuclease-free water, (closed squares), a scrambled aptamer that demonstrates no binding affinity to the SARS-CoV-2 RBD (scrambled aptamer, open squares), or the iridium nanoplate alone (Ir, open triangles). As a positive control, a subset of animals was treated with a clinically approved SARS-CoV-2 monoclonal antibody (S309, closed triangles). Animals were subsequently i.n. infected with a lethal dose of a mouse-adapted SARS-CoV-2 (1×105 PFU), two hours after treatment. Animals were monitored daily for weight loss and clinical signs of disease for 7 days, with a cohort sacrificed at 4 DPI to assess lung pathology and enumerate lung viral burden.

Animals treated with nuclease-free water, the iridium nanoplate, or scrambled aptamer exhibited rapid and continued weight loss, reaching humane endpoint (80% of starting body weight) by 4 DPI (FIGS. 18B and C). In agreement with these observations, all animals presented with significant signs of clinical disease, as marked by rapid respiration, hunched posture, and ruffled body condition (FIG. 18D). In stark contrast, TMSA52 and Ir-TMSA52 exhibited transient weight loss 2 DPI (≈10%), but quickly rebounded to pre-infection weights. No weight loss was observed in animals that received the control S309 monoclonal antibody. Neither TMSA52, Ir-TMSA52, nor S309-treated animals presented with any signs of clinical disease by 4 DPI.

To further evaluate the protection provided by i.n. aptamer delivery, lungs were harvested 4 days post-infection for both enumeration of viral burden and gross assessment of lung pathology. In accordance with both weight loss and signs of clinical disease, lungs from animals treated with either nuclease-free water, the iridium nanoplate, or scrambled aptamer exhibited areas of diffuse hemorrhage (Figure. 18E, black arrows). In stark contrast, lungs from animals treated with TMSA52 or Ir-TMSA52 showed no signs of lung pathology and were equivalent to those treated with S309.

Viral burden was subsequently quantified by a TCID50 assay. As expected, animals treated with either the mutant aptamer or the iridium nanoplate alone had similarly high lung viral titers, comparable to nuclease-free water treated control animals (FIG. 18F). In stark contrast, 2/5 animals treated with TMSA52 had a >4 log 10 reduction in lung viral burden. Of note, animals which received Ir-TMSA52 had no quantifiable lung viral titers, comparable to animals treated with S309.

### Conclusions:

Intranasal delivery of both the trimeric aptamer (TMSA52) and the aptamer scaffolded on the iridium nanoplate (Ir-TMSA52) offers robust protection against SARS-CoV-2. These treatments effectively mitigate morbidity, mortality, lung pathology, and substantially reduce lung viral burden. The performance of both TMSA52 and Ir-TMSA52 was comparable to that of the clinically approved SARS-CoV-2 monoclonal antibody S309. These findings underscore the potential of our aptamer technology as an effective prophylactic strategy against SARS-CoV-2, exhibiting promising comparability to existing clinical standards.

The disclosures of all patents, patent applications, publications and database entries referenced in this specification are hereby specifically incorporated by reference in their entirety to the same extent as if each such individual patent, patent application, publication and database entry were specifically and individually indicated to be incorporated by reference.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

1. A homotrimeric DNA aptamer comprising three aptameric nucleotide sequences selected from the group consisting of SEQ ID NOS: 1-110, a functional fragment, and a functional variant thereof; a treblar biomolecular connector comprising three arms, each arm being linked to one of the three aptameric nucleotide sequences by a linker; and a 3′ arm comprising a multipurpose attachment site on the connector molecule;
wherein the three aptameric nucleotide sequences together form a binding domain to cooperatively bind to a homotrimeric severe acute respiratory syndrome coronavirus 2 (SARS-CoV-2) spike protein, or a variant thereof, in a symmetric configuration that structurally aligns with the symmetric configuration of the homotrimeric SARS-CoV-2 spike protein, to synergistically enhance avidity for the homotrimeric SARS-CoV-2 spike protein.

2. The homotrimeric DNA aptamer of claim 1, wherein the 3′ arm is attached to a functional molecule selected from an aptamer, a reporter molecule, and a crosslinker.

3. The homotrimeric DNA aptamer of claim 2, wherein the reporter molecule is selected from an antigen, an enzyme, and a fluorescent molecule.

4. The homotrimeric DNA aptamer of claim 2 wherein the crosslinker is selected from thiol, amide, biotin, digoxigenein, azide, alkyne, carboxyl, and a click-chemistry-based crosslinker.

5. The homotrimeric DNA aptamer of claim 1, wherein the linker is a nucleotide or non-nucleotide linker.

6. The homotrimeric DNA aptamer of claim 5, wherein the linker comprises a natural or a synthetic nucleotide linker.

7. The homotrimeric DNA aptamer of claim 6, wherein the linker comprises polythymidine.

8. The homotrimeric DNA aptamer of claim 7, wherein the linker comprises 15-thymine.

9. The homotrimeric DNA aptamer of claim 1, wherein the trebler biomolecule comprises phosphoramidite.

10. The homotrimeric DNA aptamer of claim 1 wherein the aptamer can universally identify SARS-CoV-2 spike protein and variants thereof.

11. The homotrimeric DNA aptamer of claim 10 wherein the aptamer binds the SARS-CoV-2 spike protein with picomolar affinity or femtomolar affinity.

12. Use of the homotrimeric DNA aptamer of claim 1 for detecting SARS-CoV-2 spike protein and variants thereof.

13. A biosensor comprising the homotrimeric DNA aptamer of claim 1 immobilized on and/or in a material for detecting SARS-CoV-2 spike protein and variants thereof.

14. A method for detecting the presence of a SARS-CoV-2 spike protein and variants thereof in a sample, the method comprising:
a) Contacting the sample with the aptamer of claim 1, wherein the aptamer binds the SARS-CoV-2 spike protein or variants thereof; and
b) Detecting the binding of the aptamer with the SARS-CoV-2 spike protein or variants thereof;
wherein detecting the binding of the aptamer with the SARS-CoV-2 spike protein or variants thereof, comprises detecting a signal, wherein detecting of the signal indicates the presence of the SARS-CoV-2 spike protein or variants thereof in the sample.

15.-18. (canceled)

19. The method of claim 14, wherein the method detects SARS-CoV-2 infection in a subject.

20. (canceled)

21. A kit for detecting SARS-CoV-2 spike protein or variants thereof in a sample, wherein the kit comprises the biosensor of claim 13, and instructions for use of the kit.

22. A therapeutic formulation comprising the homotrimeric DNA aptamer of claim 1, and a solvent, a carrier, a diluent, and/or a dispersant.

23.-28. (canceled)

29. A method for treating a viral infection in a subject comprising administering to the subject an effective amount of the formulation according to claim 22.

30. The method according to claim 29, wherein the viral infection is SARS-CoV-2 virus.

31.-34. (canceled)

35. A kit comprising a container having contained therein the formulation according to claim 22, the container adapted to deliver the composition by an intranasal or pulmonary route.

36. (canceled)
